(19) United States
(12) Patent Application Publication
(10) Pub. No.: US 2026/0335374 A1
(43) Pub. Date: Oct. 29, 2026

(54) COMPOSITIONS AND METHODS FOR TREATING KIDNEY DISEASE

(71) Applicant: Orbsen Therapeutics Limited, Galway (IE)

(72) Inventors: Larry A. COUTURE, Claremont, CA (US); Stephen J. ELLIMAN, Raha (IE)

(21) Appl. No.: 19/118,243

(22) PCT Filed: Oct. 12, 2023
(86) PCT No.: PCT/IB2023/000587

§ 371 Date: Apr. 3, 2025

Related U.S. Application Data
(60) Provisional application No. 63/379,352, filed on Oct. 13, 2022.

(51) Int. Cl. A61K 35/35; A61P 13/12

(52) U.S. Cl. CPC A61K 35/35; A61P 13/12

(57) ABSTRACT

Provided herein are compositions and methods for treating kidney disease, for example diabetic kidney disease, using compositions comprising SDC2+ stromal stem cells.

### CROSS REFERENCE

This application claims the benefit of U.S. Provisional Application No. 63/379,352, filed Oct. 13, 2022, which is incorporated herein by reference in its entirety.

### BACKGROUND

Diabetic kidney disease or diabetic nephropathy is currently defined as chronic kidney disease secondary to diabetes and is a complication of both type 1 and type 2 diabetes. Diabetic kidney disease affects the ability of the kidneys to remove waste and excess fluid from the body. One in three diabetics in the United States have diabetic kidney disease. Over time, diabetic kidney disease can progress to kidney failure or end-stage kidney disease requiring dialysis and/or a kidney transplant.

### SUMMARY

Provided herein are methods of treating a diabetic kidney disease in an individual. In some embodiments, the method comprises administering a single dose of about 80×10⁶ SDC2+ stromal cells to the individual, thereby treating the diabetic kidney disease. In some embodiments, the single dose is sufficient to improve kidney function compared to an untreated individual. In some embodiments, the kidney function improvement is evaluated by measuring glomerular filtration rate (mGFR) or estimating glomerular filtration rate (eGFR). In some embodiments, measured glomerular filtration rate (mGFR) or estimated glomerular filtration rate (eGFR) is improved compared to a baseline measurement. In some embodiments, measured glomerular filtration rate (mGFR) or estimated glomerular filtration rate (eGFR) does not decline compared to a baseline measurement. In some embodiments, the single dose treatment is effective for at least 18 months. In some embodiments, the single dose treatment prevents kidney failure for at least 18 months. In some embodiments, frequency of regulatory T cells (CD4⁺CD25^{high}FoxP3⁺CD127⁻) is increased after the single dose treatment in the individual compared to an untreated individual. In some embodiments, frequency of memory regulatory T cells (measured by CD4⁺CD25^{high}FoxP3⁺CD127⁻CD45RA⁻CD45RO⁺ or CD4+Helios⁺CD95⁺HLA-DR⁻) is increased after the single dose treatment in the individual compared to an untreated individual. In some embodiments, the frequency of natural killer T (NKT) cells (CD3+CD56+) does not increase compared to an untreated individual. In some embodiments, the frequency of intermediate activated monocytes (HLADR⁺CD33⁺CD14⁺CD16⁺) and/or non-classical patrolling monocytes (HLADR+CD33+CD14-CD16+) is suppressed or decreased after the single dose treatment in the individual compared to an untreated individual. In some embodiments, serum cytokines NGAL and sTNFR1 are decreased after the single dose treatment in the individual compared to an untreated individual. In some embodiments, the frequency of regulatory T cells, memory regulatory T cells, NKT cells, intermediate activated monocytes and/or non-classical patrolling monocytes is measured observed for about 12 months to about 18 months after treatment. In some embodiments, the individual has type 2 diabetes or suffering from a symptom of type 2 diabetes. In some embodiments, the individual has urinary albumin excretion (UAE) of at least 60 μg/min prior to the treatment. In some embodiments, the individual has urine albumin-to-creatinine ratio (UACR) of at least 88 mg/g or 10 mg/mmol prior to the treatment. In some embodiments, the individual has an eGFR of at least 30 to 50 ml/min/1.73 m² prior to the treatment. In some embodiments, the individual has experienced a decline in eGFR of at least 10 ml/min/1.73 m² over three years prior to the treatment. In some embodiments, the individual has experienced a decline in eGFR of at least 5 ml/min/1.73 m² over 18 months prior to the treatment.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows difference in eGFR from baseline to 18 months post-treatment.

FIG. 2 shows difference in eGFR from −30 months to baseline and baseline to 18 months post-treatment.

FIG. 3A shows a study flowchart of the NEPHSTROM cohort trial.

FIG. 3B shows an overall NEPHSTROM clinical treatment plan and follow up.

FIGS. 4A-4G show frequency of peripheral blood leukocytes during the study period. Numbers are presented as a percentage within CD45+ peripheral blood leukocytes in participants randomized to ORBCEL-M or placebo during follow up. FIG. 4A shows CD4+ T cells. FIG. 4B shows CD8+ T cells. FIG. 4C shows B cells. FIG. 4D shows Lin-HLADR+ dendritic cells. FIG. 4E shows monocytes. FIG. 4F shows cytotoxic NK cells. FIG. 4G shows natural killer T cells. Values are expressed as median (IQR). *P<0.05 between the ORBCEL-M and placebo groups (ANCOVA). § P<0.05 versus preinfusion in the group (Wilcoxon test). Lin-: CD3, CD14, CD16, CD19, CD20, and CD56.

FIGS. 5A-5D show frequency of peripheral blood Tregs and Treg subpopulations during the study period. Percentages of Tregs (FIG. 5A), CD45RA−RO+ memory Tregs (FIG. 5B), Helios+CD95+HLA−DR− memory Tregs (FIG. 5C), and CD45RA+RO− naïve Tregs (FIG. 5D) within peripheral blood CD3+CD4+ T cells in participants randomized to ORBCEL-M or placebo during the follow-up. Values are expressed as median (IQR). Tregs, regulatory T cells. *P<0.05 between the ORBCEL-M and placebo groups (ANCOVA). Tregs, regulatory T cells.

FIGS. 6A-6C show frequency of peripheral blood monocyte subpopulations during the study period. Percentages of HLADR+CD33+CD14+CD16− monocytes (FIG. 6A), HLADR+CD33+CD14−CD16+ monocytes (FIG. 6B), and HLADR+CD33+CD14+CD16+ monocytes (FIG. 6C within CD45+ peripheral blood leukocytes in participants randomized to ORBCEL-M or placebo during the follow up period. Values are expressed as median (IQR). *P<0.05 between the ORBCEL-M and placebo groups (ANCOVA).

FIGS. 7A-7D show levels of proinflammatory mediators, serum concentrations of TNFR1 (FIG. 7A), NGAL (FIG. 7B), VCAM-1 (FIG. 7C), and EGF (FIG. 7D) in participants randomized to ORBCEL-M or placebo during the follow-up. Values are expressed as median (IQR). § P<0.05 versus preinfusion in the group (Wilcoxon test). TNFR1, soluble tumor necrosis factor 1; NGAL, neutrophil gelatinase-associated lipocalin; VCAM-1, vascular cell adhesion molecule 1; EGF, epidermal growth factor.

FIG. 8 shows progression of diabetic kidney disease in the two study groups. Risk of diabetic kidney disease progression in participants randomized to ORBCEL-M or placebo for change in the 2-year risk of achieving end stage kidney failure from baseline to 18 months on the basis of the validated Tangri 4-variable kidney failure risk equation. Total number of participants at baseline, ORBCEL-M n=12 and placebo n=4, and at 18 months ORBCEL-M n=10 (2 died before the end of the study) and placebo n=4.

FIGS. 9A-9D shows correlations between percentages of Tregs or CD45RA−RO+ memory Tregs and serum concentrations of inflammatory mediators in the overall study cohort. Correlations between percentages of Tregs (FIG. 9A) or CD45RA−RO+ memory Tregs (FIG. 9B) within peripheral blood CD3+CD4+ T cells and serum TNFR1 concentrations in the overall study cohort (r=−0.3690 and −0.4024, respectively; both P<0.001). Correlations between percentages of Tregs (FIG. 9C) or CD45RA−RO+ Tregs (FIG. 9D) within peripheral blood CD3+CD4+ T cells and serum NGAL concentrations in the overall study cohort (r=−0.3879 and −0.3907, respectively; both P<0.001). TNFR1, tumor necrosis factor receptor 1; NGAL, neutrophil gelatinase-associated lipocalin.

FIGS. 10A-10D show correlations between percentages of Tregs or CD45RA−RO+ memory Tregs and estimated glomerular filtration rate in the overall study cohort. Correlations between percentages of Tregs within peripheral blood CD3+CD4+ T cells and glomerular filtration rate estimated with CKD-EPI (FIG. 10A) or MDRD (FIG. 10B) equations in the overall study cohort (r=0.2235, P=0.035 and r=0.2949, P<0.005, respectively). Correlations between percentages of CD45RA−RO+ memory Tregs within peripheral blood CD3+CD4+ T cells and glomerular filtration rate estimated with CKD-EPI (FIG. 10C) or MDRD (FIG. 10D) equations in the overall study cohort (r=0.2578, P=0.015 and r=0.3313, P<0.002, respectively). CKD-EPI, Chronic Kidney Disease Epidemiology Collaboration equation; MDRD, Modification of Diet in Renal Disease equation.

FIGS. 11A-11F show correlations between serum concentrations of inflammatory mediators and measured or estimated glomerular filtration rate in the overall study cohort. Correlations between serum TNFR1 concentrations and glomerular filtration rate measured by iohexol plasma clearance (FIG. 11A) or estimated by CKD-EPI (FIG. 11B) or MDRD (FIG. 11C) equations in the overall study cohort (r=−0.3929, P=0.002, r=−0.5493, P<0.001 and r=−0.5928, P<0.001, respectively). Correlations between serum NGAL concentrations and glomerular filtration rate measured by iohexol plasma clearance (FIG. 11D) or estimated by CKD-EPI (FIG. 11E) or MDRD (FIG. 11F) equations in the overall study cohort (r=−0.5932, P<0.001, r=−0.3486, P=0.001 and r=−0.3626, P=0.001, respectively). CKD-EPI, Chronic Kidney Disease Epidemiology Collaboration equation; MDRD, Modification of Diet in Renal Disease equation; NGAL, neutrophil gelatinase-associated lipocalin; TNFR1, tumor necrosis factor receptor 1.

### DETAILED DESCRIPTION

Type 2 diabetes mellitus (DM) is a rapidly increasing global health care challenge, estimated to affect 437 million individuals worldwide in 2019. Among its complications, diabetic kidney disease (DKD) affects 30%-40% of adults living with type 2 diabetes and accounts for approximately 40% of people with end stage kidney failure (ESKF) requiring kidney replacement therapy in high-income countries. Clinically, DKD is typically characterized by the onset of microalbuminuria, which can further progress to macroalbuminuria, and a subsequent decline in GFR, ultimately leading to uremia. A wide range of maladaptive processes, predominantly driven by hyperglycemia, contribute to the pathobiology of DKD, including increased oxidative stress, chronic inflammation, accumulation of advanced glycation end products, renal hypoxia, cell apoptosis, and altered renin-angiotensin-aldosterone system activation.

Over the past few decades, medical advances have substantially improved the management of patients with DKD, thereby prolonging their survival. However, despite optimal treatments of metabolic and BP control, lipid management, and proteinuria, patients with DKD continue to have high renal and cardiovascular risk. Although recent clinical trials of sodium-glucose cotransporter 2 (SGLT2) inhibitors and other pharmacological agents have shown that the rate of renal function loss can be slowed in DKD because

of type 2 DM, successful targeting of multiple injurious pathways—such as those mediating inflammation, oxidative stress, renal hypoxia, and fibrosis—may be necessary to halt progression of DKD.

Among novel therapeutic strategies for DKD, cell therapy with MSCs is emerging as an option on the basis of its potential to deliver or induce the production of a wide range of mediators that simultaneously target maladaptive processes contributing to kidney injury. Numerous studies in preclinical models of diabetes and diabetic nephropathy have shown that MSCs exert beneficial renoprotective activities by modulating, both locally and systemically, several of the key pathophysiologic pathways that underpin DKD. Clinical translation of this cell therapy, however, has been limited, although encouraging results were reported after iv infusion of allogeneic mesenchymal precursor cells (rexlemestrocel-L) in adults with type 2 DM and moderate to-severe chronic kidney disease (CKD).

Stromal stem cells provided herein are isolated from a population of cells based on expression of the cell surface marker syndecan-2 (SDC2). Stromal stem cells are population of immunomodulatory fibroblastic cells that are isolated from one or more of human bone marrow, adipose tissue, placenta, and umbilical cord tissue. In some cases, small numbers of stromal stem cells are isolated from these tissues and cultured in vitro or ex vivo to proliferate as plastic-adherent cells, and to form colonies of fibroblasts (CFU-F). Stromal stem cells act as immune system modulators.

Stromal stem cells can, when subjected to an inflammatory and apoptosis-inducing environment containing CD95/Fas Ligands and Granzyme B/Perforins, activate flippases such as TMEM30a/CDC50a and caspases that present Phosphatidylserine (PS) to the stromal stem cell surface and cleave the Pannexin 1 channel respectively. Caspase cleavage of Pannexin-1 causes release of specific immune-metabolites including but not limited to the polyamine Spermidine, UDP, ATP, and lactate that act as “find me” signals to attract circulating Phagocytes via ATP dependent P2Y receptors. Released ATP can also be processed by approaching phagocytes expressing CD39/CD73 to produce local adenosine to dampen inflammation via adenosine receptors. This also leads to the expression of anti-inflammatory and pro-resolution molecules, such as Nr4a1, Nr4a2, arginase, and thrombospondin. Activation of TMEM30a/CDC50a presents PS to the stromal stem cell surface as an “eat me” signal to the attracted phagocytes. Efferocytosis of PS+ stromal stem cells causes inflammatory phagocytes to accumulate ingested lactate, polyamines/Spermidine, and ATP, among other metabolites. Accumulation of polyamines/Spermidine, lactate, and ATP in efferocytotic phagocytes can induce hypusination of eukaryotic translation factor 5A (eIF5A). The natural amino acid hypusine (Ne-4-amino-2-hydroxybutyl(lysine)) is derived from the polyamine spermidine, and occurs only in a single family of cellular proteins, eIF5A isoforms. Hypusine is formed by conjugation of the aminobutyl moiety of spermidine to a specific lysine residue of this protein. The posttranslational synthesis of hypusine involves two enzymatic steps catalyzed by deoxyhypusine synthase (DHPS) and deoxyhypusine hydroxylase (DOHH). Hypusine is essential for efficient eIF5A activity. Spermidine is therefore needed to hypusinate the translation factor eIF5A. Hypusinated eIF5A (eIF5A^{H}) promotes the efficient expression of a subset of mitochondrial proteins involved in the TCA cycle and oxidative phosphorylation (OXPHOS). Several of these enzymes have mitochondrial targeting sequences (MTSs), such as succinate coenzyme A ligase (SUCLG1), methylmalonyl-CoA mutase (MCM), and succinate dehydrogenase (SDHA), that in part confer an increased dependency on eIF5A^{H}. In monocyte/macrophage/phagocytes, metabolic switching between OXPHOS and glycolysis supports divergent functional fates stimulated by activation signals. In these phagocytes, hypusination of eIF5A appears to be dynamically regulated after activation driving expression of resolution proteins like Nr4a1, Nr4a2, Arginase, IDO1, and Amphiregulin. Inhibition of this hypusination blunts OXPHOS-dependent phagocyte polarization/alternative activation. Without being limited to any one theory, stromal stem cells can undergo Fas/GrB mediated apoptosis and release Spermidine/ATP via caspase activated Pannexin 1 and inflammatory phagocytes endocytose Spermidine/ATP which therapeutically controls phagocyte activation by targeting the polyamine-eIF5A-hypusine axis.

Stromal stem cells, in some cases, also secrete proteins and extracellular vesicles (exosomes) that contain significant immuno-suppressive factors such as transforming growth factor β 1 (TGFβ1), Indoleamine 2,3-dioxygenase 1 (IDO1), TNF-stimulated gene 6 (TSG6) and the purinergic enzymes CD39 and CD73. Without being limited to any one theory, using this collection of factors, stromal stem cells induce numbers of regulatory T cells, suppress proliferation of both T helper and cytotoxic T cells, decrease the production of the pro-inflammatory cytokines interferon γ (IFN-γ), tumor necrosis factor α (TNF-α) and IL-2, inhibit the activation of natural killer cells, arrest B-cell maturation, and block maturation of dendritic cells, resulting in reduced expression of antigens and co-stimulatory molecules necessary to activate T-cells. Accordingly, disclosed herein are methods of treatment of kidney disease, such as diabetic kidney disease or diabetic nephropathy.

### Methods of Treating Diabetic Kidney Disease

Provided herein are methods of treating a diabetic kidney disease in an individual. In some embodiments, the method comprises administering a single dose of about 80×10⁶ syndecan-2 positive (SDC2+) stromal cells to the individual, thereby treating the diabetic kidney disease. In some embodiments, the SDC2+ stromal cells are a population of SDC2+ stromal cells where at least 10% of the cells are SDC2+. In some embodiments, at least 15% of the cells are SDC2+. In some embodiments, at least 20% of the cells are SDC2+. In some embodiments, at least 25% of the cells are SDC2+. In some embodiments, at least 30% of the cells are SDC2+. In some embodiments, at least 35% of the cells are SDC2+. In some embodiments, at least 40% of the cells are SDC2+. In some embodiments, at least 50% of the cells are SDC2+. In some embodiments, at least 60% of the cells are SDC2+. In some embodiments, at least 70% of the cells are SDC2+. In some embodiments, at least 80% of the cells are SDC2+. In some embodiments, at least 90% of the cells are SDC2+. In some embodiments, at least 95% of the cells are SDC2+. In some embodiments, at least 99% of the cells are SDC2+. In some embodiments, 100% of the cells are SDC2+. In some embodiments, the single dose is sufficient to improve kidney function compared to an untreated individual. In some embodiments, the kidney function improvement is evaluated by measuring glomerular filtration rate (mGFR). Alternatively, or in combination, the kidney function improvement is evaluated by estimating glomerular filtration rate (eGFR). In some embodiments, mGFR is improved compared to a baseline measurement. Alternatively, or in combination, eGFR is improved compared to a baseline measurement.

In another aspect, provided herein are compositions comprising about 80×10⁶ SDC2+ stromal cells for use in treatment of diabetic kidney disease in an individual. In some embodiments, the single dose is sufficient to improve kidney function compared to an untreated individual. In some embodiments, the kidney function improvement is evaluated by measuring glomerular filtration rate (mGFR). Alternatively, or in combination, the kidney function improvement is evaluated by estimating glomerular filtration rate (eGFR). In some embodiments, mGFR is improved compared to a baseline measurement. Alternatively, or in combination, eGFR is improved compared to a baseline measurement.

In some embodiments of methods of treating diabetic kidney disease provided herein, kidney function is improved by about 5%, about 10%, about 20%, about 30%, about 40%, about 50%, about 60%, about 70%, about 80%, about 90%, about 100%, about 120%, about 150%, about 200%, or more compared to an untreated or placebo treated individual. In some embodiments, the kidney function is improved by about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 5% to about 120%, about 5% to about 150%, about 5% to about 200%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 10% to about 120%, about 10% to about 150%, about 10% to about 200%, about 20% to about 30%, about 20% to about 40%, about 20% to about 50%, about 20% to about 60%, about 20% to about 70%, about 20% to about 80%, about 20% to about 90%, about 20% to about 100%, about 20% to about 120%, about 20% to about 150%, about 20% to about 200%, about 30% to about 40%, about 30% to about 50%, about 30% to about 60%, about 30% to about 70%, about 30% to about 80%, about 30% to about 90%, about 30% to about 100%, about 30% to about 120%, about 30% to about 150%, about 30% to about 200%, about 40% to about 50%, about 40% to about 60%, about 40% to about 70%, about 40% to about 80%, about 40% to about 90%, about 40% to about 100%, about 40% to about 120%, about 40% to about 150%, about 40% to about 200%, about 50% to about 60%, about 50% to about 70%, about 50% to about 80%, about 50% to about 90%, about 50% to about 100%, about 50% to about 120%, about 50% to about 150%, about 50% to about 200%, about 60% to about 70%, about 60% to about 80%, about 60% to about 90%, about 60% to about 100%, about 60% to about 120%, about 60% to about 150%, about 60% to about 200%, about 70% to about 80%, about 70% to about 90%, about 70% to about 100%, about 70% to about 120%, about 70% to about 150%, about 70% to about 200%, about 80% to about 90%, about 80% to about 100%, about 80% to about 120%, about 80% to about 150%, about 80% to about 200%, about 90% to about 100%, about 90% to about 120%, about 90% to about 150%, about 90% to about 200%, about 100% to about 120%, about 100% to about 150%, about 100% to about 200%, about 120% to about 150%, about 120% to about 200%, or about 150% to about 200%.

In another aspect of methods of treatment herein, kidney function does not decline compared to a baseline measurement. In some embodiments, mGFR does not decline compared to a baseline measurement. In some embodiments, eGFR does not decline compared to a baseline measurement. In some embodiments, kidney function does not decline more than about 1%, about 2%, about 5%, about 10%, or about 20% compared to a baseline measurement. In some embodiments, kidney function is maintained at about 90%, about 95%, about 99%, or about 100% compared to a baseline measurement.

In another aspect of methods of treatment herein, the single dose treatment is effective for at least about 6 months, about 12 months, about 18 months, about 24 months, about 36 months, or longer. In some embodiments, the single dose treatment prevents kidney failure for at least about 6 months, about 12 months, about 18 months, about 24 months, about 36 months, or longer.

In another aspect of methods of treatment provided herein, frequency of one or more immune cells are altered in a treated individual compared with an untreated individual or an individual treated with placebo. In some embodiments, frequency of regulatory T cells (CD4+CD25highFoxP3+CD127−) is increased after the single dose treatment in the individual compared to an untreated individual. In some embodiments, frequency of memory regulatory T cells (CD4+CD25highFoxP3+CD127−CD45RA−CD45RO+ or CD4+Helios⁺CD95⁺HLA-DR⁻) is increased after the single dose treatment in the individual compared to an untreated individual. In some embodiments, the frequency of natural killer T (NKT) cells (CD3+CD56+) does not increase compared to an untreated individual. In some embodiments, the frequency of intermediate activated monocytes (HLADR⁺CD33⁺CD14⁺CD16⁺) and/or non-classical patrolling monocytes (HLADR+CD33+CD14−CD16+) is suppressed after the single dose treatment in the individual compared to an untreated individual. In some embodiments, alteration in the frequency of one or more immune cells is observed for about 12 months to about 18 months after treatment.

In some embodiments, the serum cytokines NGAL and sTNFR1 are decreased after the single dose treatment in the individual compared to an untreated individual.

In another aspect of methods of treatment provided herein, the individual has type 2 diabetes. In some embodiments, the individual is suffering from a symptom of type 2 diabetes. In some embodiments, the individual has urinary albumin excretion (UAE) of at least 60 μg/min prior to the treatment. In some embodiments, the individual has urine albumin-to-creatinine ratio (UACR) of at least 88 mg/g or 10 mg/mmol prior to the treatment. In some embodiments, the individual has an eGFR of at least 30 to 50 ml/min/1.73 m² prior to the treatment. In some embodiments, the individual has experienced a decline in eGFR of at least 10 ml/min/1.73 m² over three years prior to the treatment. In some embodiments, the individual has experienced a decline in eGFR of at least 5 ml/min/1.73 m² over 18 months prior to the treatment.

### Preparation of Stromal Stem Cells for Therapeutic Use

Stromal stem cells for therapeutic are isolated from human umbilical cord tissue (UCT) or bone marrow by selecting for cells expressing CD362 (SDC2).

Methods of preparing stromal stem cells for treatment of diabetic kidney disease herein, in some cases comprise obtaining an umbilical cord tissue sample. Often, the method comprises sterilizing the umbilical cord sample. In some cases, the method comprises dividing the umbilical cord sample into a known weight. Often, the method comprises further cutting each portion of umbilical cord into small pieces. In some cases, the method comprises cutting each portion of umbilical cord into pieces no larger than 0.1 mm², 0.2 mm², 0.3 mm², 0.4 mm², 0.5 mm², 0.6 mm², 0.7 mm², 0.8 mm², 0.9 mm², or 1.0 mm². In some cases, the method comprises cutting each portion of umbilical cord into pieces about 0.5 to about 1 mm\hat{ }2. In some cases, the method comprises removing any blood from the umbilical cord samples. In some cases, the method comprises mixing the pieces of umbilical cord with a protease, such as collagenase, trypsin, proteinase K, or other protease. Often, the umbilical cord samples are incubated with a protease for at least 30, 35, 40, 45, 50, 55, or 60 minutes. In some cases, the method comprises stopping the protease reaction with a cell culture media comprising serum. Often the method comprises filtering the protease treated umbilical cord samples through a cell strainer, such as a 100 μm cell strainer, resulting in a solution comprising the cells from the umbilical cord.

Method of preparing stromal stem cells for treatment of diabetic kidney disease herein comprise labeling the dissociated umbilical cord cells with an agent that binds to SDC2, such as an anti-SDC2 antibody. In some cases, cells labeled with the anti-SDC2 cell antibody are separated from the unlabeled umbilical cord cells. In some cases, labeled cells are separated from unlabeled cells using fluorescence activated cell sorting. In some cases, labeled cells are separated from unlabeled cells using a magnetic cell separating device. In some cases, labeled cells are separated from unlabeled cells using a MACSQuant Tyto device. Often, dead cells are removed from the cells during the cell separation step. In some cases, the cell separation step comprises an enrichment sort. In some cases, the cell separation step comprises an enrichment sort and a purity sort. In some cases, the sorted cells are counted. Often, the method comprises culturing the sorted cells to expand cell numbers.

Stromal stem cells for treatment of diabetic kidney disease are, in some cases, mixed with an excipient and stored prior to use. In some cases, the excipient comprises a cryoprotectant or cryopreservative. Cryoprotectants include, but are not limited to DMSO, glycerol, polyethylene glycol, propylene glycol, glycerine, polyvinylpyrolidone, sorbitol, dextran, trehalose, and commercial formulations such as CryoStor from Biolife solutions. Stromal stem cell compositions herein retain potency after being frozen using special freezing protocols. Special freezing protocols include flash freezing, programmable rate freezer, and freezing in an insulated container. The stromal stem cell compositions are in some cases frozen in buffer or culture media having an added cryoprotectant. Buffers include physiologically acceptable buffers such as phosphate buffer, histidine buffer, citrate buffer, acetate buffer, Hypothermasol from Biolife Solutions and other suitable.

### DEFINITIONS

“Stromal stem cells”, “mesenchymal stem cells”, “SDC2+ stromal stem cells”, “ORBCEL-M™” or “ORBCEL-C™”, used interchangeably are SDC2+ cells isolated from umbilical cord tissue or bone marrow having therapeutic properties such as treating inflammatory diseases, such as inflammatory liver diseases, and wounds, such as non-healing wounds.

“SDC2,” also known as syndecan-2, CD362, S2, or fibroglycan, refers generally herein to the SDC2 polypeptide encoded by the SDC2 locus. Syndecan-2, or ‘the SDC2 protein’ or simply SDC2, is a transmembrane type I heparin sulfate proteoglycan. Additional synonyms for syndecan-2, aside from ‘the SDC2 protein’ or SDC2, include HSPG, CD362, HSPG1, and SYND2. Generally, as used herein SDC2 refers to the protein or a recognizable fragment thereof unless otherwise indicated, for example by reciting ‘the SDC2 gene,’ ‘the SDC2 transcript,’ ‘an SDC2 antibody.’ An SDC2 fragment refers to any set of consecutive residues of SDC2 that uniquely or recognizably map to the SDC2 polypeptide sequence. In some cases an SDC2 fragment retains some or all activity of the SDC2 protein, or acts as an inhibitor of full length or native SDC2. SDC2 also occasionally refers informally herein to the locus or gene encoding the SDC2 protein. In the event that one of skill in the art is unable to distinguish an SDC2 reference, it is presumed that the term is used herein in reference to the protein or polypeptide rather than to the gene, transcript, or an antibody raised against or binding to SDC2. There is a family of syndecan proteins in mammals. SDC2 is used alternately in reference to a mammalian syndecan-2 or to human SDC2 specifically. In the event that one of skill in the art is unable to distinguish an SDC2 reference, it is presumed that the term is used herein in reference to the human protein or polypeptide.

The terms “recipient”, “individual”, “subject”, “host”, and “patient”, are used interchangeably herein and in some cases, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. Sometimes, the mammal is human.

As used herein, the terms “treatment,” “treating,” “ameliorating a symptom,” and the like, in some cases, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject. An “untreated individual” refers to an individual who has a kidney disease (e.g., diabetic kidney disease) and/or is suffering from a symptom of the kidney disease (e.g., diabetic kidney disease), and has received a placebo treatment or alternatively refers to an individual has a kidney disease (e.g., diabetic kidney disease) and/or is suffering from a symptom of the kidney disease (e.g., diabetic kidney disease), and who has not been treated at all.

The terms “pharmaceutically acceptable”, “physiologically tolerable” and grammatical variations thereof, as they refer to compositions, carriers, diluents, and reagents, are used interchangeably and in some cases, represent that the materials are capable of administration to or upon a human without the production of undesirable physiological effects to a degree that would prohibit administration of the composition.

A “therapeutically effective amount” in some cases means the amount that, when administered to a subject for treating a disease, is sufficient to effect treatment or ameliorate a symptom of that disease.

A “baseline measurement” in some cases refers a quantitative or qualitative measurement before a treatment is provided or begins.

As used herein, the term “about” a number refers to a range spanning that from 10% less than that number through 10% more than that number, and including values within the range such as the number itself.

As used herein, the term “comprising” an element or elements of a claim refers to those elements but does not preclude the inclusion of an additional element or elements.

“Diabetic kidney disease” or “diabetic nephropathy” used interchangeably herein, refers to a complication of type 1 and type 2 diabetes caused by damage to blood vessel clusters in the kidneys that filter waste from blood. This leads to persistent albuminuria, progressive decline in glomerular filtration rate, and elevated arterial blood pressure.

“About” a number, as used herein, refers to range including the number and ranging from 10% below that number to 10% above that number. “About” a range refers to 10% below the lower limit of the range, spanning to 10% above the upper limit of the range.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Treatment of Diabetic Kidney Disease

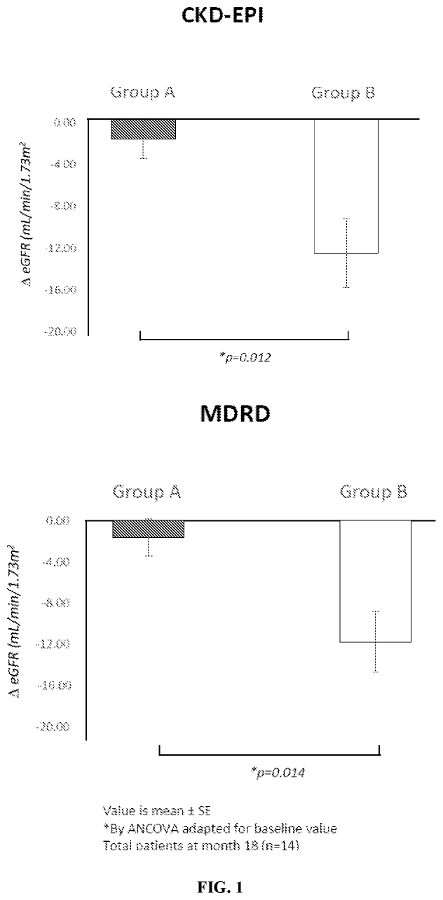

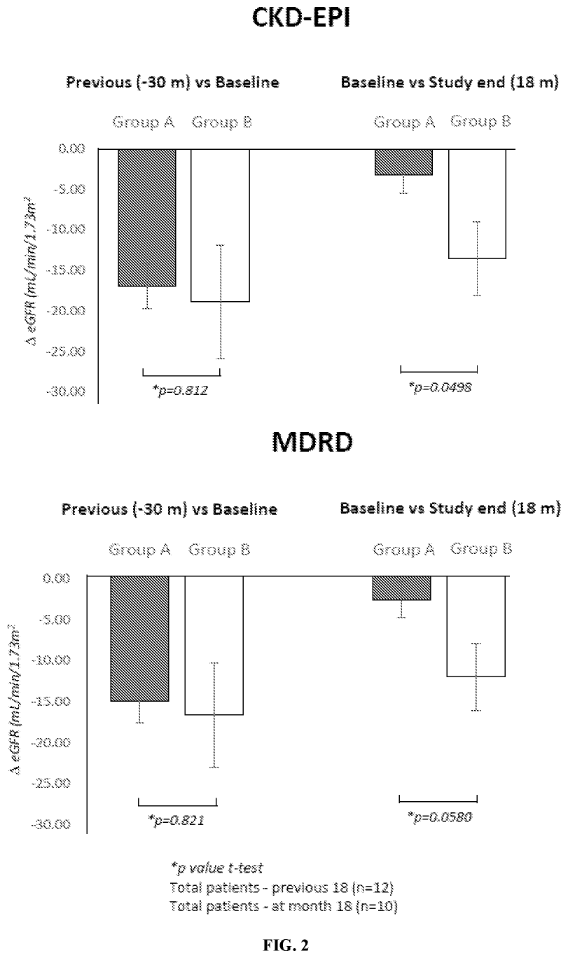

Patients with type 2 diabetes and progressive diabetic kidney disease were infused with a single dose of 80×10⁶ SDC2+ stromal stem cells isolated from bone marrow (ORBCEL-M) or placebo and then monitored for 18 months. At baseline, 6 months, 12 months, and 18 months, measured glomerular filtration rates (mGFR) and estimated glomerular filtration rates (eGFR) were assessed. Data for mGFR at baseline, 6 months, 12 months, and 18 months is shown in Table 1. Data for eGFR at baseline, 6 months, 12 months, and 18 months is shown in Table 2. The difference in eGFR from baseline to 18 months post treatment is shown in FIG. 1. FIG. 2 shows difference in eGFR from −30 months to baseline and baseline to 18 months. Annual rate of renal function decline from baseline to 18 months follow up is shown in Table 3. A selection of metabolic parameters (Table 4), arterial blood pressure and heart rate (Table 5), and urine albumin-creatinine ration (UACR) were also studied over the course of the study. ORBCEL-M treated individuals showed a significantly smaller decline in eGFR from baseline to 18 months post treatment (FIG. 1, FIG. 2, and Tables 2 and 3).

TABLE 1
Time course of GFR during 18 month follow up in patients receiving IMP (80 × 10⁶ cells)/ Placebo

| | | ORBCEL- | | | | |
|---|---|---|---|---|---|---|
| | | M | Difference vs | Placebo | Difference vs | |
| | | mean | Baseline mean | mean | Baseline mean | P value** |
| | | value | (range) | value | (range) | A vs B |
| mGFR | Baseline | 45.77 | | 44.59 | | |
| (ml/min) | 6 months | 42.78 | −2.55 (−8.6-3.49) | 41.05 | −5.78 (−22.79-11.23) | 0.613 |
| | 12 months | 40.95 | −2.96 (−8.18-2.27) | 37.70 | −6.89 (−28.49-14.71) | 0.496 |
| | 18 months | 39.46 | −6.94 (12.38-1.51) | 33.72 | −10.87 (−37.01-15.27) | 0.286 |
| mGFR | Baseline | 37.83 | | 37.89 | | |
| (ml/min/ | 6 months | 34.82 | −1.79 (−6.63-3.04) | 36.12 | −4.35 (16.20-7.5) | 0.709 |
| 1.73 m²) | 12 months | 33.10 | −2.53 (−6.38-1.35) | 31.33 | −6.50 (−25.32-12.32) | 0.443 |
| | 18 months | 31.86 | −5.51 (−9.89-1.13) | 28.11 | −9.72 (−32.00-12.55) | 0.236 |

mGFR, measured glomerular filtration rate by plasma clearance of iohexol
**By ANCOVA adjusted for baseline value

TABLE 2
Time course of estimated GFR during 18 month follow up
in patients receiving IMP (80 × 10⁶ cells)/ Placebo

| | | ORBCEL- | | | | |
|---|---|---|---|---|---|---|
| | | M | Difference vs | Placebo | Difference vs | |
| | | mean | Baseline mean | mean | Baseline mean | P value** |
| | | value | (range) | value | (range) | A vs B |
| eGFR | Baseline | 36.35 | | 36.00 | | |
| (CDK- | 6 months | 36.16 | 0.65 (−4.2, −5.50) | 32.19 | −5.58 (−13.27, −2.11) | 0.239 |
| EPI) | 12 months | 35.55 | −0.90 (−3.43, 1.62) | 28.82 | −7.18 (−13.06, −1.31) | 0.015 |
| | 18 months | 35.00 | −1.88 (−6.04, −2.28) | 23.23 | −12.78 (−23.22, −2.31) | 0.012 |
| eGFR | Baseline | 36.52 | | 35.01 | | |
| (MDRD) | 6 months | 36.46 | 0.64 (−4.06, −5.34) | 31.49 | −5.11 (−11.92, −1.69) | 0.226 |
| | 12 months | 35.93 | −0.73 (−3.19, −1.73) | 28.43 | −6.58 (−11.87, −1.29) | 0.018 |
| | 18 months | 35.42 | −1.66 (−5.73, −2.40) | 23.21 | −11.80 (−21.18, −2.42) | 0.014 |

eGFR, estimated glomerular filtration rate by CKD-EPI or MDRD equation
**By ANCOVA adjusted for baseline value

TABLE 3
Annual rate of renal function decline from baseline to 18 months follow-up

| | ORBCEL-M | | |
|---|---|---|---|
| | median | Placebo median | P value** |
| mGFR (ml/mn per year) | −4.3 [−7.3, −2.3] | −8.2 [−15.3, +1.3] | 0.467 |
| mGFR (ml/min/1.73 m² per year) | −3.8 [−5.7, −2.1] | −7.5 [−13.9, +1.0] | 0.467 |
| eGFR/CKD-EPI (ml/min/1.73 m² per | −2.6 [−4.2, −0.3] | −8.7 [011.4, −4.6] | 0.034 |
| year) | | | |
| eGFR/MDRD (ml/min/1.73 m² per | −2.4 [−4.0, −0.1] | −8.1 [−10.4, −4.4] | 0.034 |
| year) | | | |

**Wilcoxon rank sum ORBCEL-M vs Placebo
Values are median [IQR]

TABLE 4
Time course of selective metabolic parameters

| | ORBCEL- | Difference vs | Placebo | Difference vs | P |
|---|---|---|---|---|---|
| | M mean | Baseline mean | mean | Baseline mean | value** |
| | value | (range) | value | (range) | A vs B |
| Glucose (mg/dL) | | | | | |
| Baseline | 142.23 | | 176.55 | | |
| 6 months | 150.28 | −9.23 (−23.52-5.06) | 143.47 | −18.73 (−64.01-26.54) | 0.323 |
| 12 months | 134.87 | −8.36 (−24.89-8.17) | 140.60 | −35.95 (−87.93-16.03) | 0.482 |
| 18 months | 143.88 | 4.49 (−17.40-26.38) | 158.5 | −18.05 (−55.07-18.97) | 0.826 |
| HbA1c (nmol/mol) | | | | | |
| Baseline | 57.53 | | 55.35 | | |
| 6 months | 55.44 | −2.09 (−9.08-4.90) | 49.35 | −1.5 (−29.46-26.47) | 0.971 |
| 12 months | 54.59 | −5.25 (−13.12-2.52) | 52.25 | −3.10 (−23.70-17.50) | 0.940 |
| 18 months | 57.91 | −0.99 (−7.41-5.43) | 55.63 | 0.27 (−28.51-29.06) | 0.959 |
| Total Cholesterol (mg/dL) | | | | | |
| Baseline | 169.98 | | 119.42 | | |
| 6 months | 158.94 | −9.62 (−27.34-8.10) | 118.71 | −0.71 (−10.87-9.45) | 0.645 |
| 12 months | 165.20 | −7.57 (−31.51-16.35) | 122.37 | 2.49 (−38.56-43.33) | 0.507 |
| 18 months | 160.63 | −9.93 (−38.99-19.14) | 121.50 | 0.04 (−12.98-13.07) | 0.695 |
| LDL Cholesterol (mg/dL) | | | | | |
| Baseline | 93.94 | | 51.24 | | |
| 6 months | 83.32 | −10.62 (−37.61-16.36) | 47.95 | −3.29 (−10.85-4.26) | 0.850 |
| 12 months | 96.73 | 1.13 (−24.95-27.21) | 55.54 | 1.29 (−14.61-17.19) | 0.957 |
| 18 months | 84.12 | −8.82 (−39.20-21.55) | 37.50 | −1.24 (−18.37-15.89) | 0.988 |
| Triglycerides (mg/dL) | | | | | |
| Baseline | 205.58 | | 100.72 | | |
| 6 months | 185.88 | −28.73 (−66.23-8.78) | 121.85 | 21.13 (−52.63-94.89) | 0.619 |
| 12 months | 166.73 | −42.25 (−90.51-6.01) | 130.44 | 20.10 (−91.90-132.10) | 0.919 |
| 18 months | 180.10 | −35.37 (−68.80-−1.95) | 202.14 | 30.15 (−104.00-164.30) | 0.282 |
| CRP (mg/dL) | | | | | |
| Basal | 0.38 | | 0.53 | | |
| 6 months | 1.07 | 0.67 (−1.11-2.45) | 0.29 | −0.23 (−1.25-0.78) | 0.625 |
| 12 months | 0.47 | 0.15 (−0.41-0.70) | 0.74 | 0.16 (−0.92-1.23) | 0.642 |
| 18 months | 0.25 | −0.09 (−0.41-0.23) | 0.30 | −0.27 (−1.16-0.62) | 0.996 |

**By ANCOVA adjusted for baseline value

TABLE 5
Time course of arterial blood pressure and heart rate

| | ORBCEL− | Difference vs | Placebo | Difference vs | P |
|---|---|---|---|---|---|
| | M mean | Baseline mean | mean | Baseline mean | value** |
| | value | (range) | value | (range) | A vs B |
| SBP (mmHg) | | | | | |
| Baseline | 136.08 | | 136.25 | | |
| 6 months | 144.08 | 8.00 (−3.03-19.03) | 151.33 | 17.67 (0.22-35.11) | 0.421 |
| 12 months | 137.18 | 1.45 (−11.72-19.61) | 134.50 | −1.75 (−14.77-11.27) | 0.761 |
| 18 months | 135.40 | −0.40 (−15.64-14.84) | 135.25 | −1.00 (−13.86-11.86) | 0.968 |
| DBP (mmHg) | | | | | |
| Baseline | 71.83 | | 78.00 | | |
| 6 months | 74.75 | 2.92 (−5.76-11.60) | 79.33 | 1.33 (−15.58-18.24) | 0.580 |
| 12 months | 72.36 | 1.18 (−7.53-9.89) | 76.00 | −2.00 (−11.63-7.63) | 0.673 |
| 18 months | 68.20 | −3.50 (−9.65-2.65) | 73.50 | −4.50 (−12.35-3.35) | 0.812 |
| MAP (mmHg) | | | | | |
| Baseline | 93.25 | | 97.42 | | |
| 6 months | 97.86 | 4.61 (−3.25-12.38) | 103.33 | 6.78 (−10.22-23.77) | 0.377 |
| 12 months | 93.97 | 1.27 (−8.12-10.66) | 95.50 | −1.92 (−11.33-7.50) | 0.572 |
| 18 months | 90.60 | −2.47 (−10.06-5.13) | 94.08 | −3.33 (−12.25-5.58) | 0.835 |
| Heart rate (Bit/min) | | | | | |
| Baseline | 67.42 | | 70.75 | | |
| 6 months | 66.00 | −1.42 (−5.40-2.57) | 67.67 | −6.33 (−24.98-12.31) | 0.375 |
| 12 months | 68.55 | 0.82 (−7.21-8.85) | 70.50 | −0.25 (−11.97-11.47) | 0.899 |
| 18 months | 68.40 | 0.60 (−5.52-6.72) | 74.50 | 3.75 (−19.92-26.42) | 0.461 |

**By ANCOVA adjusted for baseline value

TABLE 6
Time course of UACR during 18 month follow up

| | ORBCEL− | | | | |
|---|---|---|---|---|---|
| | M | Difference vs | Placebo | | |
| | mean | Baseline | mean | Difference vs Baseline | P value** |
| | value | mean (range) | value | mean (range) | A vs B |
| UACR (mg/g) | | | | | |
| Baseline | 1337.69 | | 2165.79 | | |
| 6 months | 1365.85 | 33.80 (−719.9-859.5) | 2821.02 | 327.4 (−441.8-1096.6) | 0.421 |
| 12 months | 1086.55 | 235.1 (−278.9-749.0) | 2280.64 | 114.9 (−921.6-1151.3) | 0.543 |
| 18 months | 1084.65 | 132.1 (−198.4-462.7) | 1759.06 | −406.7 (−1188.9-375.4) | 0.161 |

**By ANCOVA adjusted for baseline value

### Example 2: Safety and Efficacy of ORBCEL-M Therapy in Diabetic Kidney Disease

A phase 1b/2a multicenter, randomized, placebo-controlled clinical trial (NEPHSTROM trial) was conducted with the primary aim of investigating the safety and feasibility, and a secondary aim of preliminary

assessment of efficacy, of cell therapy with a next-generation human bone marrow-derived, antibody-purified (CD3621) population of MSCs (ORBCEL-M) in individuals with type 2 DM with progressive DKD. In this study, results of the first ORBCEL-M dose/placebo cohort recruited into the NEPHSTROM trial are reported.

### Methods

### Trial Design and Participants

The NEPHSTROM (Novel Stromal Cell Therapy for Diabetic Kidney Disease) study is a pilot, exploratory, investigator-initiated, European, multicenter, randomized, double-blind, placebo-controlled clinical trial. It was performed at three academic clinical centers in Ireland (University of Galway), Italy (Azienda Socio-Sanitaria Territoriale Papa Giovanni XXIII, ASST-PG23, Bergamo), and the United Kingdom (University Hospital Birmingham NHS Foundation Trust, UHBFT, Birmingham) and was coordinated by the Istituto di Ricerche Farmacologiche Mario Negri IRCCS (IRFMN), Bergamo, Italy. A second clinical center in the United Kingdom (Belfast Health and Social Care Trust, BHSCT, Belfast), initially working as a clinical trial enrollment site, withdrew, but remained in the NEPHSTROM study as the centralized laboratory for screening and monitoring of anti-HLA antibodies in the study participants. The local ethics committees and the national competent authorities approved the study protocol and the Investigational Medicine Product (IMP). For these regulatory approvals, the NEPHSTROM consortium followed the Voluntary Harmonization Procedure ([VHP1038][VHP2017011]). The trial was registered with the European Union Clinical Trial Register (EUDRACT No 2016-000661-23) and at ClinicalTrials.gov (NCT02585622). Written informed consent was obtained from all participants.

Eligible participants were between 40 and 85 years with type 2 DM for 3 or more years under a clinician with mandated responsibility for management to national guidelines. Other inclusion criteria were as follows: (1) urine albumin-to creatinine ratio (UACR)≥88 mg/g (≥10 mg/mmol) in a spot morning urine sample; (2) eGFR 25-55 ml/min per 1.73 m² by the Chronic Kidney Disease Epidemiology Collaboration (CKD-EPI) equation on two or more consecutive measurements at least 30 days apart within the past 6 months; and (3) a documented eGFR decline of ≥10 ml/min per 1.73 m² over the past 3 years or documented rate of eGFR decline≥5 ml/min per 1.73 m² per year on the basis of 3 or more consecutive readings at least 90 days apart, within the past 18 months up to the date of consent, or an intermediate or high 5-year risk of progression to ESKF (dialysis or transplantation) on the basis of the validated Tangri 4-variable (age, sex, eGFR, urinary albumin/creatinine ratio) kidney failure risk equation for patients with CKD stage 3-5. Key exclusion criteria were (1) resting systolic BP≥150 mm Hg and diastolic BP≥90 mm Hg in a clinical setting, despite treatment with three antihypertensive agents of different classes; (2) hemoglobin A1c (HbA1c)>75 mmol/mol (>9%); (3) fasting total cholesterol>7 mmol/L; (4) fasting total triglycerides>3.5 mmol/L; and (5) positive screening test for anti-HLA antibodies (mean fluorescence intensity>1500). Patients with chronic lung or liver disease, with cardiovascular events within 6 months before enrollment, currently enrolled or had a history within 6 months before enrollment of New York Heart Association (NYHA) class III or IV heart failure, and with active malignancy or women of childbearing potential without use of acceptable methods of contraception or women who were pregnant or lactating were also excluded.

In keeping with the large majority of therapeutic trials of interventions for DKD, none of the participants enrolled in the NEPHSTROM trial had a kidney biopsy at study entry to confirm the presence of pathological changes of diabetic nephropathy and to rule out non-DKDs. One of the 16 enrolled participants had a previously recorded kidney biopsy (which confirmed pathological abnormalities consistent with diabetic nephropathy), and one had a prior biopsy attempt which did not yield diagnostic tissue.

### Procedures and Assessments

The NEPHSTROM trial follows a phase 1b/2a dose-escalation design aiming to recruit 48 participants with type 2 diabetes and DKD who have provided written consent. Equal numbers of participants were expected to be enrolled by each center. However, if difficulty in enrollment was to occur in one or more centers, additional recruitment could be implemented in the other participating centers. Study participants were randomly allocated in a 3:1 ratio to a double-blind single iv infusion of one of three ORBCEL-M doses (80, 160, or 240×10⁶ cells) or to placebo infusion. Each of the three cohorts consists of 16 participants (12 receiving ORBCEL-M and 4 receiving placebo [Cryostor CS10]). Because the NEPHSTROM trial is a preliminary safety study, the first cohort of participants received the lowest dose (80×10⁶ cells) or placebo.

If the Data Monitoring Safety Board indicated that the study could proceed beyond this dose, the next 16 participants could have been allocated and randomized to the next ORBCEL-M 160×10⁶ cell dose or placebo in the absence of a dose-limiting toxicity event (cohort 2). Finally, after completion of allocation to cohort 2, the subsequent 16 participants would have been allocated to the last 240×10⁶ cell dose or placebo (cohort 3). As detailed herein, cohort 2 enrollment was ended prematurely (13 of 16 participants) after discussion among the principal investigators and the study sponsor because of the coronavirus disease 2019 (COVID-19) pandemic, which precluded any further activities related to the NEPHSTROM trial. Moreover, cohort 3 was not performed because of the delay in the trial from the COVID-19 pandemic and the material inability to further extend validation of cell/placebo bags to be used for iv infusion.

Active and placebo treatments were administered in the context of ongoing independent standard-of-care medical management of glycemia, BP, lipid levels, and other clinical issues by specialist physicians blinded to treatment randomization.

At each clinical center, patients with type 2 diabetes were prescreened for potential eligibility by trained study personnel on the basis of ongoing outpatient evaluation and medical record review. After obtaining informed consent from participants who fulfilled inclusion criteria and agreed to participate in the study, screening tests were performed to confirm suitability for randomization. Prerandomization tests consisted of basic blood parameters, UACR, serum anti-HLA antibody screen (Luminex bead assay), and, for women of childbearing potential, a pregnancy test. Participants confirmed as meeting eligibility criteria were then randomized to ORBCEL-M or placebo infusion according to a computer-generated randomization procedure through the Clinical Trial Coordinating Center, IRFMN. The randomization list was prepared by an independent statistician (Giovanni Antonio Giuliano) at the Laboratory of Biostatistics of Clinical Research Center for Rare Diseases Aldo e Cele Daccò, IRFMN (Ranica, Bergamo, Italy).

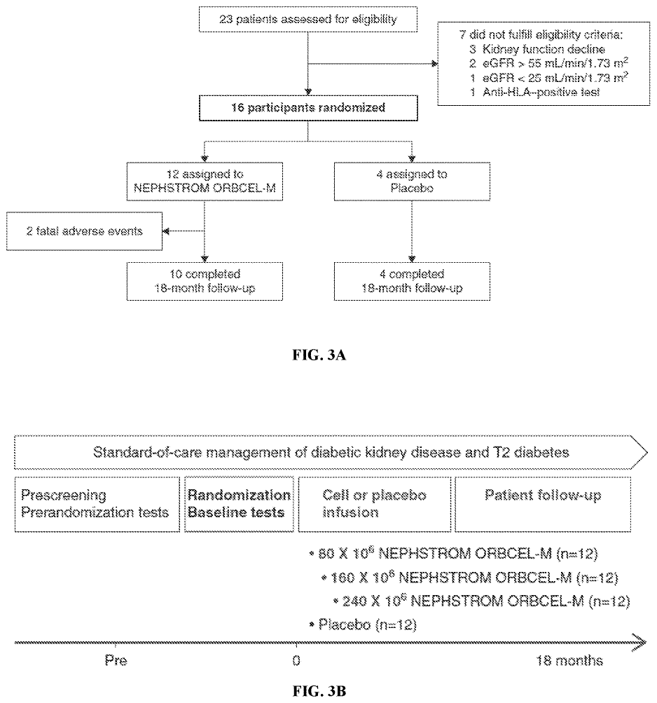

Each randomized participant was admitted to a clinical research facility (CRF) for baseline evaluations, including systolic and diastolic BP, electrocardiogram, fasting blood glucose, HbA1c, lipid profile, and hematology and biochemistry panels with GFR estimation by CKD-EPI and Modification of Diet in Renal Disease (MDRD) equations. UACR was measured on spot morning urine samples; GFR was measured by plasma iohexol clearance. Blood and urine samples were also processed and stored for profiling of blood immune cell subsets, and biomarkers of inflammation. The trial infusion (ORBCEL-M or placebo) was administered within 48 hours (FIG. 3B). The participants were closely monitored during the infusion and thereafter for an 8-hour period in the CRF.

On days 1 and 7 and at month 1 after infusion, the participants returned to the CRF, where they were interviewed regarding symptoms and adverse events (AEs) (early post-IMP/placebo administration monitoring). At these time points, they had measurements of BP, serum creatinine, fasting blood glucose, hematology and biochemistry panels, and UACR. At the 7-day and 1-month time points, blood and urine samples were collected and processed for immune monitoring and assays of inflammation-related soluble mediators.

Subsequent CRF follow-up visits at 3, 6, 12 and 18 months after ORBCEL-M/placebo infusion included physical examination, interviews for intercurrent AEs, and routine laboratory tests (later postadministration monitoring). At 6, 12, and 18 months, kidney function (GFR by plasma iohexol clearance, and eGFR by CKD-EPI and MDRD equations) was assessed, UACR measured on spot morning urine samples, and blood collected for immune/inflammatory mediator monitoring. Trial follow-up was ended at 18 months after infusion.

The trial imposed no restrictions on concomitant treatments, which were at the discretion of the participants primary and specialist physicians. At randomization and at each follow-up visit thereafter, concomitant treatments were reviewed and recorded.

### ORBCEL-M Preparation, Administration, and Postinfusion Monitoring

ORBCEL-M was manufactured from healthy donor bone marrow aspirates under license (Orbsen Therapeutics Ltd, Galway, Ireland) by three Good Manufacturing Practice (GMP) facilities (Centro di Terapia Cellulare Gilberto Lanzani, ASST-PG23, Bergamo, Italy; Center for Cellular Manufacturing CCMI, Galway, Ireland; NHS Blood and Transplant NHSBT, Liverpool, United Kingdom), with a fourth GMP facility—Academish Ziekenhuis Leiden-Leids Universitair Medisch Centrum LUMC, Leiden, the Netherlands—serving as the primary isolation and coordinating site responsible for the manufacturing protocol. Bone marrow aspirates were collected from screened, healthy adult volunteers by a consultant hematologist at the Irish HPRA-approved Galway Blood and Tissue Establishment and were shipped to Leiden University Medical Center. Here, ORBCEL-M cells were antibody-enriched in a CliniMACS separation system using a GMP-grade anti-CD362 antibody and were primarily expanded to passage (P)1 in tissue culture flasks. The P1 cells were lifted, using recombinant enzyme; reseeded into a cryoprecipitate-coated hollow-fiber bioreactor (Quantum Cell Expansion System [Terumo BCT Europe N.V., Belgium]) where they were further expanded for a maximum of 7 days; and collected by enzymatic release. Aliquots of cells were cryopreserved as an intermediate ORBCEL-M product and shipped to the other three GMP teams for a subsequent second round of expansion in a Quantum Cell Expansion System bioreactor, followed by harvest, formulation at the required doses into individual cryobags, and final release of the GMP product. The GMP facilities of Leiden, Bergamo, Galway, and Liverpool each hold a national license for the manufacturing of Advanced Therapy Medicinal Products, including MSC products. On the day of infusion, GMP-released doses of ORBCEL-M or placebo, cryopreserved in 40-ml sterile bags, were transported frozen to the CRFs in Bergamo, Galway, and Birmingham. The GMP facilities released ORBCEL-M according to specific criteria, including (1) cell surface marker positivity≥95% by FACS analysis for CD73, CD90, and CD105, as well as ≤1% positivity for CD45 and CD34; (2) negative for mycoplasma, gram-positive and gram-negative bacteria, and fungi; (3) endotoxin below 10 EU/ml by a chromogenic method; (4) viability≥70% by trypan blue and manual counting; (5) viable cell number≥350×10⁶; and (6) karyotype G banding and Q banding analysis (no clonal abnormalities and no more than three individual abnormalities). This conforms with the criteria for defining multipotent MSCs by the International Society of Cellular Therapy.

Immediately after thawing and according to randomization, the ORBCEL-M or placebo dose (in a 40-ml volume) was administered intravenously into a peripheral arm vein of each randomized participant over 10-20 minutes using a 200-mm transfusion filter. A premedication regimen was administered consisting of oral acetaminophen (1 g, 1 hour before infusion) and iv chlorphenamine and hydrocortisone (10 and 100 mg, respectively, immediately before infusion). Baseline temperature, pulse rate, BP, respiratory rate, oxygen saturation, and chest auscultation were recorded and thereafter monitored continuously during the infusion, every 15 minutes during the first hour, and hourly during the subsequent 7 hours after infusion. Study participants were also monitored closely for other signs of adverse reactions, such as rash, urticaria, or wheezing. All events were recorded during the 8-hour observation period, after which the participant was allowed to leave the CRF if no AEs had occurred. The doses of ORBCEL-M and placebo were indistinguishable in labeling and instructions for use, but to avoid identification of the active IMP because of cloudiness of cell suspensions, the primary trial physicians and research nurses, as well as the participants were shielded from seeing the infusion bag and tubing to remain blinded throughout the trial while validation, thawing, and setup of the infusions were performed by a separate (unblinded) team consisting of a technician, pharmacist, and research nurse.

### Trial Outcomes

The primary trial outcome was the number and severity of prespecified cell infusion-associated events and the overall number and frequency of AEs and unexpected severe AEs during the early (up to 1 month) and late (from 2 to 18 months) follow-up periods among ORBCEL-M recipients compared with placebo recipients. Secondary outcomes included the efficacy of ORBCEL-M compared with placebo to slow the progression of DKD, assessed using the following variables: (1) GFR changes (AGFR and slope of GFR decline, evaluated by the serial measurements of mGFR by plasma iohexol clearance); (2) serum creatinine-based eGFR by CKD-EPI and MDRD equations (AeGFR and slope of eGFR decline); and (3) absolute and percentage change of UACR on spot urine samples from baseline to month 18 after infusion. Other secondary outcomes were the effects of ORBCEL-M compared with placebo on other relevant clinical parameters, including proportions of study participants within target ranges for glycemic control (fasting blood glucose and HbA1c), lipid control (total cholesterol, LDL cholesterol, and triglycerides), and BP control. Finally, additional secondary outcomes included the effect of ORBCEL-M compared with placebo on immune and inflammatory profiles, assessed by the following variables: (1) anti-HLA antibody development; (2) proportion/total number of circulating lymphocyte (T cells, B cells, and NK cells) and myeloid cell (monocytes and dendritic cells) subsets; and (3) plasma/serum immunoassay-derived concentrations of biomarkers of inflammation.

### Laboratory Measurements

Clinical chemistry was performed according to a trial monitoring protocol. Blood and urine samples were analyzed by local clinical laboratories at the three participating centers. Measured GFR (mGFR) was determined by an established protocol for quantifying the plasma clearance of unlabeled iohexol.34 For this, series of plasma samples were collected and initially stored at each CRF at −80 °C and then were shipped for centralized measurement to the Laboratory of the Clinical Trial Coordinating Center, IRFMN, Ranica, Italy. Iohexol plasma levels were assayed by high-performance liquid chromatography. Measurement of anti-HLA antibodies in serum were also centralized at the Belfast HSC Trust Histocompatibility and Immunogenetics Laboratory, Belfast, United Kingdom. The procedure consisted of two Luminex assays: an initial antibody screen with Luminex multiantigen beads to detect class I and class II MHC antibodies, followed, if necessary, by a Luminex single-antigen bead assay to determine the specificity of any antibody detected. Longitudinal profiling of peripheral blood lymphocyte and myeloid cell subsets were centralized at the Laboratory of Immunology and Organ Transplantation, IRFMN, at the Clinical Trial Coordinating Center, Italy. Three antibody panels were used to analyze the phenotype of (1) CD4+ and CD8+ T-cell subsets, B-cell subsets, NK, and monocytes; (2) regulatory CD4+ T cells (Tregs); and (3) Lin-HLADR+ dendritic cells by the FACS LSR Fortessa X-20 (Becton Dickinson) and FlowJo software. For the inflammatory biomarker evaluation, longitudinal samples collected in the three participating clinical centers were centralized at University of Galway, Ireland. Serum concentrations of soluble tumor necrosis factor receptor 1 (sTNFR1), neutrophil gelatinase-associated lipocalin (NGAL), vascular cell adhesion molecule (VCAM-1), and epidermal growth factor (EGF) (biomarkers with well-documented links to DKD severity

and prognosis) were quantified by using DuoSet ELISA kits (R&D Systems, Minneapolis) according the manufacturer's instructions.

### Sample Size Estimation

Although this is a phase 1 study with the primary objective of determining feasibility and safety, a sample size was calculated according to Cocks and Torgerson for the initial efficacy of ORBCEL-M in slowing the rate of decline of GFR. It was determined that the study should include at least 36 participants to be powered to detect a change in the rate of decline of GFR from 5.1 (SD 4.3) ml/min per year in the placebo group to 3.4 ml/min per year in the active group (power=80% and α=0.05, two-sided test). This calculation was based on previously reported interim data in study participants with diabetes enrolled in the Preventing ESRD in Overt Nephropathy of Type 2 Diabetes Preventing ESRD in Overt Nephropathy of Type 2 Diabetes (VALID) trial and taking into account the 3:1 random allocation. Thus, 48 consenting participants with type 2 DM and with progressive DKD were planned to be recruited.

### Statistical Analyses

Safety and efficacy analyses were predetermined to be primarily performed on an “all-treated” basis—i.e., to include all participants randomized into the study who received an infusion of ORBCEL-M or placebo, regardless of whether an infusion was initiated. In addition, a secondary safety analysis was also to be performed on a “safety set” basis—i.e., to include only participants randomized into the study who had received some or all of an ORBCEL-M or placebo infusion. All statistical analyses were conducted using SAS version 9.4 (SAS Institute Inc., Cary, NC) and STATA version 15 (StataCorp., College Station, TX). A mixed-effect model with random intercepts was used to determine the mGFR and eGFR slopes and was estimated by restricted maximum likelihood. Results were expressed as mean±SD or median (interquartile range) or number (%) as appropriate. All P values were two-sided with significance assigned to P<0.05.

### Results

### Participant Enrollment and Baseline Characteristics

The study protocol was planned to include three cohorts: cohort 1, 80×10⁶ cells or placebo; cohort 2, 160×10⁶ cells or placebo; and cohort 3, 240×10⁶ cells or placebo. The results presented here relate to the enrollment, treatment, and completed follow-up of cohort 1. The detailed rationale for unblinding and analyzing the data for this cohort is described elsewhere herein.

Between March 2018 and January 2020, 23 consenting patients were screened for final eligibility, of which 16 were randomized to cohort 1 of the trial. Of the seven screening failures, three were due to a failure to meet the criteria for eGFR decline, two were due to screening eGFR>55 ml/min per 1.73 m², one was due to screening eGFR<25 ml/min per 1.73 m², and one was due to a positive anti-HLA screening assay (see study flowchart, FIG. 3A). The number of participants randomized and treated at each of the sites was Bergamo, Italy, n=8; Galway, Ireland, n=4; and Birmingham, United Kingdom, n=4.

Table 7 summarizes key demographic, clinical, and laboratory characteristics at baseline of the 16 randomized Participant, according to treatment with ORBCEL-M or placebo. The median age was 69 (interquartile range, 66-73) years in the cell-treated group and 59 (interquartile range, 54-66) years in the placebo group. All participants were male. Systolic and diastolic BP were well-controlled with antihypertensive treatment in both groups. In keeping with the inclusion criteria, both groups of participants had comparable moderate-to-severe CKD on the basis of both mGFR and eGFR.

Glycemic control as determined by HbA1c was closely comparable for the two groups, and median values for fasting blood glucose, lipid parameters, and UACR were not significantly different. Only one of 16 participants was prescribed a SGLT2 inhibitor at the time of randomization.

TABLE 7
Participant baseline, demographical, clinical, and laboratory characteristics

| Parameter | ORBCEL-m (n = 12) | Placebo (n = 4) | P Value^{a} |
|---|---|---|---|
| Age (yr) | 69 (66-73) | 59 (54-66) | 0.054 |
| Sex (M/F) (n) | 12/0 | 4/0 | — |
| BP | | | |
| Systolic (mm Hg) | 137 (128-147) | 139 (129-144) | 0.144 |
| Diastolic (mm Hg) | 70 (66-78) | 79 (72-84) | 0.715 |
| Serum creatinine (mg/dl) | 1.90 (1.78-2.04) | 2.07 (1.80-2.19) | 0.379 |
| eGFR (CDK-EPI) (ml/min per 1.73 m²) | 35.3 (32.4-38.6) | 33.4 (31.9-40.0) | 0.683 |
| eGFR (MDRD (ml/min per 1.73 m²) | 35.4 (32.6-38.4) | 32.9 (31.2-38.8) | 0.379 |
| mGFR (ml/min per 1.73 m²) | 38.5 (32.4-42.6) | 39.4 (25.5-50.2) | 0.953 |
| HbA1c (mmol/mol) | 54.1 (47.6-66.8) | 56.1 (45.2-65.5) | 0.861 |
| Fasting glucose (mg/dl) | 131 (117-158) | 183 (138-215) | 0.305 |
| Total cholesterol (mg/dl) | 180 (135-201) | 106 (86-166) | 0.101 |
| UACR (mg/g) | 420.5 (289.4-2042.6) | 982.5 (470.8-3860.8) | 0.845 |
| Medication, n (%) | | | |
| Antihypertensive medication use | | | |
| ACEi | 5 (41.7) | 2 (50) | 1.000 |
| ARB | 4 (33.3) | 1 (25) | 1.000 |
| β-blocker | 6 (50) | 1 (25) | 0.585 |
| Calcium channel blockers | 6 (50) | 1 (25) | 0.585 |
| Diuretics | 5 (41.7) | 1 (25) | 1.000 |
| Metformin | 6 (50) | 2 (50) | 1.000 |
| Insulin | 12 (100) | 4 (100) | — |
| Statin | 9 (75) | 4 (100) | 0.529 |
| SGLT2i | 1 (9.1) | 0 (0) | 1.000 |

Data are median (interquartile range), unless otherwise indicated. eGFR, estimated GFR; CKD-EPI, Chronic Kidney Disease Epidemiology Collaboration; MDRD, Modification of Diet in Renal Disease; mGFR, measured GFR; HbA1c, hemoglobin Alc; UACR, urinary albumin-to-creatinine ratio; ACEi, angiotensin-converting enzyme inhibitor; ARB, angiotensin receptor blocker; SGLT2i, sodium-glucose cotransporter 2 inhibitor.
^{a}Wilcoxon test or Fisher exact test, as appropriate.

### Primary Outcome

Of the 16 randomized participants, 14 completed 18-month follow-up per protocol while follow-up of 2 (both in the ORBCEL-M-treated group) ended early because of SAEs resulting in death (described in more detail below).

### Early Safety Monitoring

Per protocol, all randomized participants were in stable health at the time of study treatment. For 15 of 16 participants, no adverse reactions to cell or placebo infusion occurred. Thus, temperature, pulse rate, respiratory rate, BP, and oxygen saturation remained stable during the infusion and the subsequent 8-hour observation period. In one placebo-treated participant, an episode of moderate bronchospasm occurred shortly after completion of the infusion. In this case, the participant recovered fully approximately 50 minutes later after appropriate pharmacologic treatment. No other adverse reactions occurred between the time of infusion and the 1-month follow-up visit

TABLE 8
Early (from 0 to 1 month) and late (from 2 to 18 months) serious adverse events

| | ORBCEL- | | | Relationship | | |
|---|---|---|---|---|---|---|
| Parameter | M/ Placebo | Sex | Event | with treatment | Intensity | Outcome |
| Early | | | | | | |
| SAEs | | | | | | |
| | Placebo | Male | Bronchospasm | Yes | Moderate | Recovered |
| Late SAEs | | | | | | |
| | ORBCEL- | Male | Acute myocardial | No | Severe | Recovered |
| | M | | infarction | | | with |
| | | | | | | sequelae |
| | | | Congestive heart | No | Severe | Fatal |
| | | | failure | | | |
| | ORBCEL- | Male | COVID-19 test | No | Mild | Recovered |
| | M | | positive | | | |
| | ORBCEL- | Male | Anemia with increased | No | Moderate | Recovered |
| | M | | dyspnea | | | |
| | | | Left hip fracture | No | Severe | Unknown |
| | | | Respiratory tract | No | Moderate | Recovered |
| | | | infection | | | |
| | | | Complicated duodenal | No | Moderate | Unknown |
| | | | diverticulitis | | | |
| | | | Respiratory failure | No | Severe | Unknown |
| | | | Hyperkalemia | No | Severe | Recovered |
| | | | Multiple myeloma | No | Severe | Fatal |
| | ORBCEL- | Male | Headache | No | Moderate | Recovered |
| | M | | | | | |

SAE, serious adverse event; COVID-19, coronavirus disease 2019.

### Late Safety Monitoring

As summarized in Table 8, eleven additional SAEs occurred in a total of four participants (all recipients of cell infusions) between months 2 and 18 of the follow-up period: seven in a single participant, two in another participant, and one each in two others. None of the late SAEs were deemed to be related to the trial investigational product, ORBCEL-M. For two of these participants, the non-treatment-related SAEs culminated in death: in one case from congestive heart failure after 15 months of trial follow-up and in the second from multiple acute complications of newly diagnosed multiple myeloma after 15 months of follow-up.

Overall, in the cell-treated group, 56 AEs were recorded, of which 11 were deemed serious (Table 9). In the placebo-treated group, 13 AEs occurred, of which one was deemed serious and treatment-related (as described above). The 57 AEs that were not classified as serious are summarized in Table 10.

TABLE 9
Summary of adverse events

| Events | ORBCEL-M | Placebo |
|---|---|---|
| Participants with at least one AE | 12 | 4 |
| Any AE | 56 | 13 |
| SAEs | 11 | 1 |
| AEs leading to death | 2 | 0 |
| AEs leading to discontinuation of study drug | 0 | 0 |
| AEs leading to discontinuation of study | 2 | 0 |
| Drug-related AE | 0 | 0 |
| Drug-related SAEs | 0 | 1 |

Data are reported as numbers. Any AE: both not serious and serious adverse events. AE, adverse event; SAE, serious adverse event

TABLE 10
List of nonserious adverse events

| | ORBCEL-M | Placebo |
|---|---|---|
| | events, n | Events, n |
| Adverse event type | (Participants, n) | (Participants, n) |
| Left bundle block | 2 (2) | — |
| First-degree atrioventricular | 1 (1) | — |
| block | | |
| Inferior Q wave | 1 (1) | — |
| Ejection systolic murmur | 1 (1) | — |
| Chest pain | 2 (2) | — |
| Fatigue | 1 (1) | — |
| Asthenia | 1 (1) | — |
| Increased edema | 4 (1) | — |
| Hypoglycemia | 1 (1) | — |
| Eye disorder | — | 1 (1) |
| Cataract | 1 (1) | — |
| Diarrhea | 3 (3) | 1 (1) |
| Heartburn | 1 (1) | — |
| Vomiting associated with ileus | 1 (1) | — |
| Hemorrhoids | — | 1 (1) |
| Fever of unknown origin | — | 1 (1) |
| Flu-like syndrome | 1 (1) | — |
| Bruised ribs | 1 (1) | — |
| Stenosing tenosynovitis | 1 (1) | — |
| Laceration to left shin | 1 (1) | — |
| Left knee swelling | 1 (1) | — |
| Traumatic injury left foot | — | 1 (1) |
| Fall | 1 (1) | — |
| Meralgia paresthetica | 1 (1) | — |
| Wound infection | | 1 (1) |
| Pain associated with SAE hip | 1 (1) | — |
| fracture | | |
| Pain right heel | | 1 (1) |
| Knee pain | 1 (1) | — |
| Painful shoulder | 1 (1) | — |
| Headache | 1 (1) | — |
| Low mood | 1 (1) | — |
| Memory impairment | 1 (1) | — |
| Disoriented | 1 (1) | — |
| Cough | 2 (2) | 1 (1) |
| Hoarse | 1 (1) | — |
| Obstructive sleep apnea | — | 1 (1) |
| Abdominal bruising | — | 1 (1) |
| Perianal dermatitis | — | 1 (1) |
| Pressure Sore (sacral) | 1 (1) | — |
| Tingling in limb peripheries | 1 (1) | — |
| Benign prostatic hyperplasia | 1 (1) | — |
| Secondary hyperparathyroidism | 1 (1) | — |
| Erythroderma | — | 1 (1) |
| Urinary tract infection | 1 (1) | — |
| AKI | 1 (1) | — |
| Hypereosinophilia | 1 (1) | — |
| Total | 45 (11) | 12 (4) |

SAE, serious adverse event

### Predefined Secondary Comparisons

### Effects on Renal Function

As summarized in Table 11, baseline mGFR (by plasma iohexol clearance) was comparable in the ORBCEL-M and placebo-treated groups. In both groups, mGFR declined during the 18-month follow-up period. The mean changes in mGFR at 6, 12, and 18 months compared with baseline were numerically less in ORBCEL-M-treated participants than in placebo-treated participants, but the differences were not statistically different (P=0.709, P=0.443, and P=0.236, respectively). By contrast, the mean changes in eGFR, whether calculated by CKD-EPI or MDRD equations, were significantly less in the ORBCEL-M group than the placebo group (at 12 months, P=0.015 and P=0.018 for CKD-EPI and MDRD, respectively; at 18 months, P=0.012 and P=0.014, respectively). Very similar results were observed when changes in renal function during trial follow-up were calculated as rate of decline per year (Table 12). For mGFR, the annual rate of decline was numerically but not significantly lower for the group receiving cells compared with the group receiving placebo (P=0.467) while for eGFR, a significantly lower annual rate of decline occurred in ORBCEL-M-treated participants than in placebo-treated participants (P=0.034 for both CKD-EPI and MDRD eGFR).

TABLE 11
Time course of mGFR and eGFR during the 18-month follow
up period in participants receiving ORBCEL-M or placebo

| | | | | | |
|---|---|---|---|---|---|
| | | Difference versus | | Difference versus | |
| | ORBCEL-M | Baseline Mean | Placebo | Baseline Mean | |
| Parameter | Mean ± SD | (Range) | Mean ± SD | (Range) | P Value^{a} |

mGFR (ml/min per 1.73 m²)

| | | | | | |
|---|---|---|---|---|---|
| Baseline | 37.24 ± 8.39 | | 37.84 ± 14.78 | | |
| 6 mo | 34.82 ± 9.91 | 21.79 (26.63 to 3.04) | 36.12 ± 12.26 | −4.35 (−16.20 to 7.5) | 0.709 |
| 12 mo | 33.10 ± 8.85 | 22.53 (26.38 to 1.32) | 31.33 ± 5.09 | −6.50 (−25.32 to 12.32) | 0.443 |
| 18 mo | 31.86 ± 6.66 | 25.51 (29.89 to 21.13) | 28.11 ± 4.74 | −9.72 (−32 to 12.55) | 0.236 |

eGFR (CDK-EPI) (ml/min per 1.73 m²)

| | | | | | |
|---|---|---|---|---|---|
| Baseline | 36.35 ± 5.44 | | 36.00 ± 7.06 | | |
| 6 mo | 36.16 ± 7.79 | 0.65 (24.20 to 5.50) | 32.19 ± 7.14 | −5.58 (−13.27 to 2.11) | 0.239 |
| 12 mo | 35.55 ± 6.57 | −0.90 (−3.43 to 1.62) | 28.82 ± 6.53 | −7.18 (−13.06 to −1.31) | 0.015 |
| 18 mo | 35.00 ± 8.85 | −1.88 (26.04 to 2.28) | 23.23 ± 4.33 | −12.78 (−23.22 to −2.31) | 0.012 |

eGFR (MDRD (ml/min per 1.73 m²)

| | | | | | |
|---|---|---|---|---|---|
| Baseline | 36.52 ± 5.27 | | 35.01 ± 6.16 | | |
| 6 mo | 36.46 ± 7.62 | 0.64 (−4.06 to 5.34) | 31.49 ± 6.58 | −5.11 (−11.92 to 1.69) | 0.226 |
| 12 mo | 35.93 ± 6.42 | −0.73 (−3.19 to 1.73) | 28.43 ± 6.05 | −6.58 (−11.87 to −1.29) | 0.018 |
| 18 mo | 35.42 ± 8.43 | −1.66 (−5.73 to 2.40) | 23.21 ± 4.23 | −11.80 (−21.18 to −2.42) | 0.014 |

mGFR, measured GFR; eGFR, estimated GFR; CKD-EPI, Chronic Kidney Disease Epidemiology Collaboration; MDRD, Modification of Diet in Renal Disease.
^{a}By ANCOVA adjusted for baseline value. ORBCEL-M versus Placebo, as difference versus baseline.

TABLE 12
Annual rate of renal function decline from baseline to 18-
month follow-up in participants receiving ORBCEL-M or placebo

| Parameter | ORBCEL-M | Placebo | P value^{a} |
|---|---|---|---|
| mGFR (ml/min per 1.73 m²) | −3.8 (−5.7 to −2.1) | −7.5 (−13.9 to 1.0) | 0.467 |
| eGFR (CDK-EPI) (ml/min per 1.73 | −2.6 (−4.2 to −0.3) | −8.7 (−11.4 to −4.6) | 0.034 |
| m²) | | | |
| eGFR (MDRD (ml/min per 1.73 m²) | −2.4 (−4.0 to −0.1) | −8.1 (−10.4 to −4.4) | 0.034 |

Data are reported as median (interquartile range). mGFR, measured GFR; eGFR, estimated GFR; CKD-EPI, Chronic Kidney Disease Epidemiology Collaboration; MDRD, Modification of Diet in Renal Disease.
^{a}Wilcoxon rank-sum test: NEPHSTROM ORBCEL-M versus placebo

Trends in UACR during trial follow-up are summarized in Table 13. As shown, UACR values varied widely in both groups at baseline and at 6, 12, and 18-month follow-ups without significant between-group differences at any time point. Notably, despite the lower rate of eGFR decline for the group receiving cells, there was no evidence of a reduction in UACR in ORBCEL-M-treated participants.

TABLE 13
Time course of urinary albumin-to-creatinine
ratio during the 18-month follow-up period
in participants receiving ORBCEL-M or placebo

| UACR (mg/g) | ORBCEL-M | Placebo | P value^{a} |
|---|---|---|---|
| Baseline | 420.5 (289.4-2042.6) | 982.5 (470.8-3860.8) | 0.845 |
| 6 mo | 1250.2 (239.0-1955.3) | 836.7 (423.3-7203.0) | 1.000 |
| 12 mo | 956.3 (403.8-1580.6) | 636.0 (333.6-4227.7) | 0.845 |
| 18 mo | 1004.9 (371.2-1317.4) | 682.8 (273.7-3244.4) | 0.832 |

Data are reported as median (interquartile range). UACR, urine albumin-to-creatinine ratio.
^{a}Wilcoxon rank-sum test: NEPHSTROM ORBCEL-M versus placebo

### Effect on Metabolic Parameters and BP

Values for blood glucose, HbA1c, serum total cholesterol, serum triglycerides, and serum C-reactive protein at baseline and after 6, 12, and 18 months of follow-up are summarized for the two groups in Table 14 along with their mean changes from baseline at the three follow-up time points. As shown, these parameters remained generally stable and within clinically acceptable ranges in both groups throughout the trial with no differences observed. Similar observations were made for BP parameters and heart rate (Table 15).

TABLE 14
Time course of metabolic parameters during the 18-month follow-up
period in participants receiving ORBCEL-M or placebo

| | | | | | |
|---|---|---|---|---|---|
| | | Difference versus | | Difference versus | |
| | ORBCEL-M | Baseline Mean | Placebo | Baseline Mean | |
| Parameter | Mean ± SD | (Range) | Mean ± SD | (Range) | P value^{a} |

Glucose (mg/dl)

| | | | | | |
|---|---|---|---|---|---|
| Baseline | 142 ± 41 | | 177 ± 47 | | |
| 6 mo | 150 ± 68 | 8 (−23 to 39) | 143 ± 74 | −19 (−64 to 27) | 0.323 |
| 12 mo | 135 ± 40 | −11 (−33 to 11) | 141 ± 38 | −34 (−88 to 16) | 0.482 |
| 18 mo | 144 ± 44 | −1 (−31 to 29) | 159 ± 44 | −18 (−55 to 19) | 0.826 |

HbA1c (nmol/mol)

| | | | | | |
|---|---|---|---|---|---|
| Baseline | 58 ± 13 | | 55 ± 12 | | |
| 6 mo | 55 ± 16 | −2 (−9 to 5) | 49 ± 14 | −2 (−29 to 26) | 0.971 |
| 12 mo | 55 ± 11 | −5 (−13 to 3) | 52 ± 9 | −3 (−24 to 18) | 0.940 |
| 18 mo | 58 ± 13 | −1 (−7 to 5) | 56 ± 12 | 0.27 (−29 to 29) | 0.959 |

Total cholesterol (mg/dl)

| | | | | | |
|---|---|---|---|---|---|
| Baseline | 170 ± 39 | | 119 ± 42 | | |
| 6 mo | 159 ± 48 | −10 (−27 to 8) | 119 ± 46 | −1 (−11 to 9) | 0.645 |
| 12 mo | 165 ± 57 | −8 (−32 to 16) | 122 ± 46 | 2 (−39 to 43) | 0.507 |
| 18 mo | 161 ± 60 | −10 (−39 to 19) | 121 ± 33 | 0.04 (−13 to 13) | 0.695 |

Triglycerides (mg/dl)

| | | | | | |
|---|---|---|---|---|---|
| Baseline | 206 ± 108 | | 101 ± 59 | | |
| 6 mo | 186 ± 86 | −29 (−66 to 9) | 122 ± 78 | 21 (−53 to 95) | 0.619 |
| 12 mo | 167 ± 62 | −42 (−91 to 6) | 130 ± 41 | 20 (−92 to 132) | 0.919 |
| 18 mo | 180 ± 90 | −35 (−69 to −2) | 202 ± 145 | 30 (−104 to 164) | 0.282 |

C-reactive protein (mg/dl)

| | | | | | |
|---|---|---|---|---|---|
| Baseline | 0.38 ± 0.47 | | 0.54 ± 0.31 | | |
| 6 mo | 1.07 ± 2.64 | 0.67 (−1.11 to 2.45) | 0.29 ± 0.20 | −0.23 (−1.25 to 0.78) | 0.625 |
| 12 mo | 0.47 ± 0.69 | 0.15 (−0.41 to 0.70) | 0.74 ± 0.31 | 0.16 (−0.92 to 1.23) | 0.642 |
| 18 mo | 0.25 ± 0.15 | −0.09 (−0.41 to −0.23) | 0.30 ± 0.10 | −0.27 (−1.16 to 0.62) | 0.996 |

^{a}By ANCOVA adjusted for baseline value. ORBCEL-M versus Placebo, as difference versus baseline.

TABLE 15
Time course of arterial BP and heart rate during the 18-month
follow-up period in participants receiving ORBCEL-M or placebo

| | | | | | |
|---|---|---|---|---|---|
| | | Difference versus | | Difference versus | |
| | ORBCEL-M | Baseline Mean | Placebo | Baseline Mean | |
| Parameter | Mean ± SD | (Range) | Mean ± SD | (Range) | P value^{a} |

SBP (mm Hg)

| | | | | | |
|---|---|---|---|---|---|
| Baseline | 136 ± 12 | | 136 ± 10 | | |
| 6 mo | 144 ± 17 | 8 (−3 to 19) | 151 ± 15 | 18 (0 to 35) | 0.421 |
| 12 mo | 137 ± 16 | 1 (−12 to 20) | 134 ± 9 | −2 (−15 to 11) | 0.761 |
| 18 mo | 135 ± 23 | 0 (−16 to 15) | 135 ± 12 | −1 (−14 to 12) | 0.968 |

DBP (mm Hg)

| | | | | | |
|---|---|---|---|---|---|
| Baseline | 72 ± 9 | | 78 ± 10 | | |
| 6 mo | 75 ± 9 | 3 (−6 to 12) | 79 ± 14 | 1 (−16 to 18) | 0.580 |
| 12 mo | 72 ± 9 | 1 (−8 to 10) | 76 ± 9 | −2 (−12 to 8) | 0.673 |
| 18 mo | 68 ± 7 | −3 (−10 to 3) | 73 ± 15 | −4 (−12 to 3) | 0.812 |

MAP (mm Hg)

| | | | | | |
|---|---|---|---|---|---|
| Baseline | 93 ± 8 | | 97 ± 9 | | |
| 6 mo | 98 ± 7 | 5 (−3 to 12) | 103 ± 13 | 7 (−10 to 24) | 0.377 |
| 12 mo | 94 ± 8 | 1 (−8 to 11) | 96 ± 8 | −2 (−11 to 7) | 0.572 |
| 18 mo | 91 ± 8 | −2 (−10 to 5) | 94 ± 13 | −3 (−12 to 6) | 0.835 |

Heart rate (beats/min)

| | | | | | |
|---|---|---|---|---|---|
| Baseline | 67 ± 13 | | 71 ± 20 | | |
| 6 mo | 66 ± 13 | −1 (−5 to 3) | 68 ± 17 | −6 (−25 to 12) | 0.375 |
| 12 mo | 69 ± 9 | 1 (−7 to 9) | 70 ± 13 | 0 (−12 to 11) | 0.899 |
| 18 mo | 68 ± 15 | 1 (−5 to 7) | 74 ± 8 | 4 (−19 to 26) | 0.461 |

SBP, systolic BP; DBP, diastolic BP; MAP, mean arterial pressure.
^{a}By ANCOVA adjusted for baseline value. ORBCEL-M versus Placebo, as difference versus baseline

### Effects on Immunological and Inflammatory Parameters

Screening for anti-HLA class I and class II antibodies was negative at all scheduled visits for 15 of 16 participants. One participant treated with ORBCEL-M tested positive for low-level anti-HLA class I antibodies starting at 12 months after infusion, which persisted to the end of trial follow-up without any clinically relevant consequences.

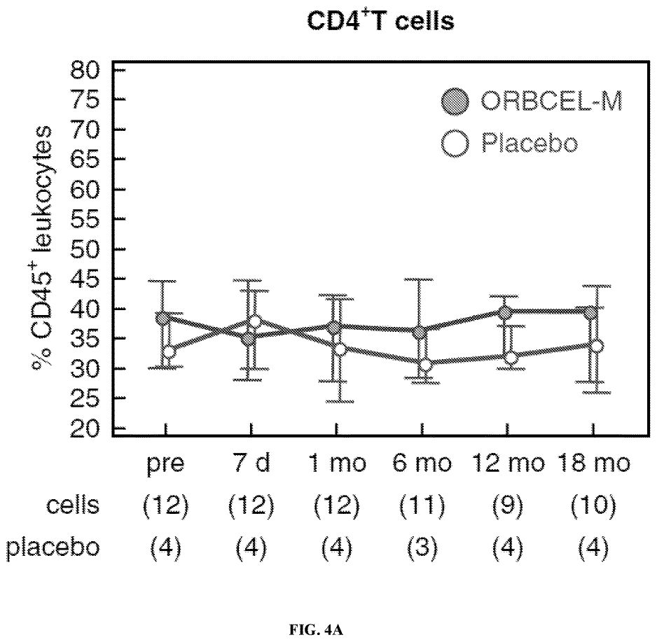

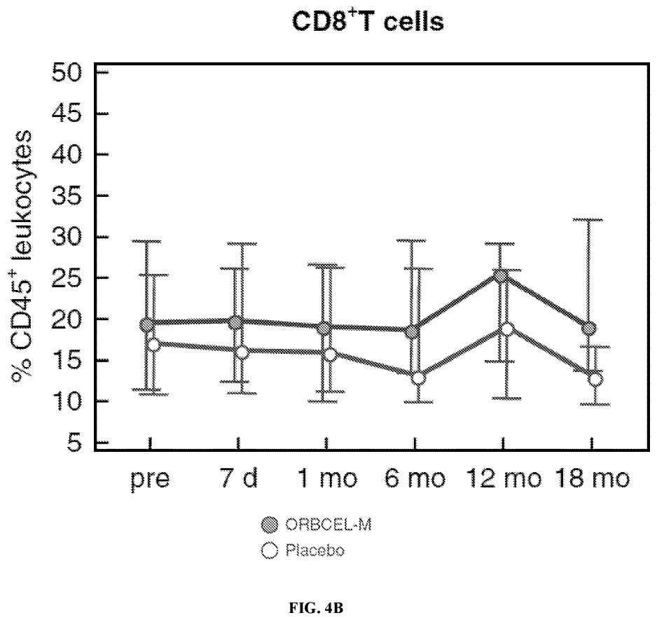

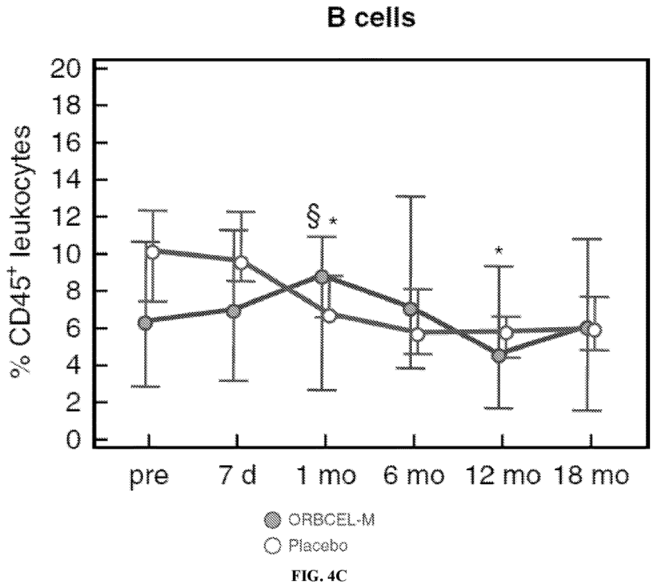

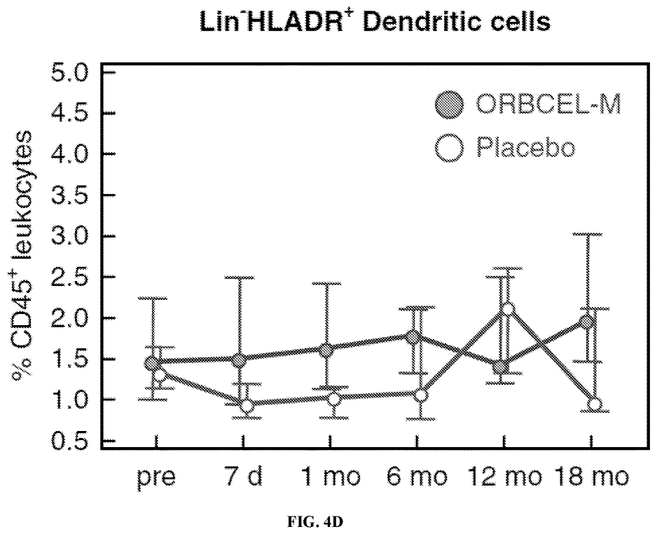

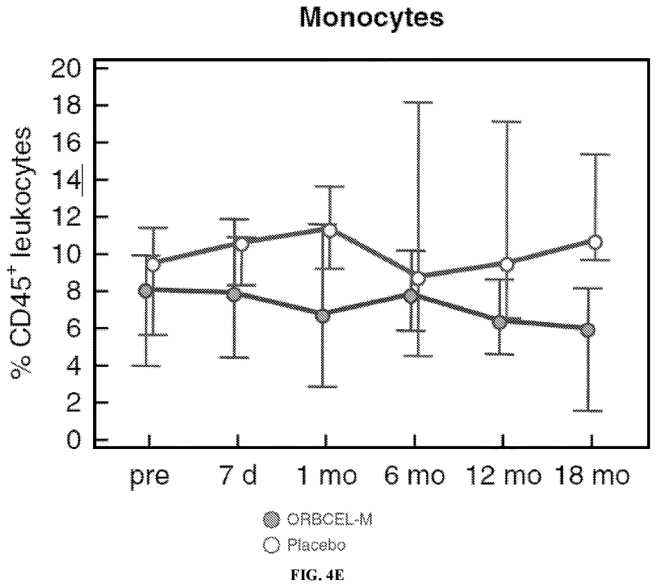

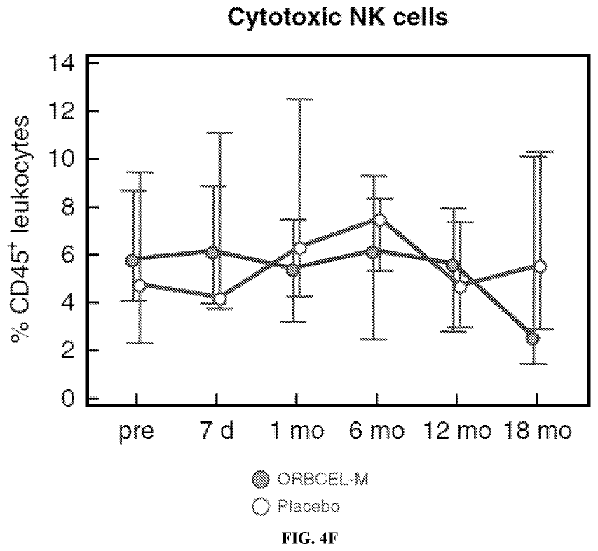

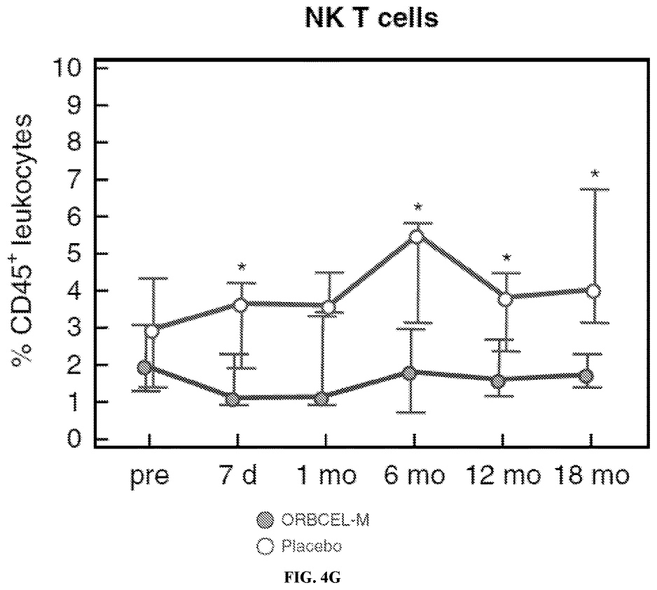

Regarding longitudinal PBMC profiling by flow cytometry, no significant changes or between-group differences were observed in the proportions of CD4+ and CD8+ T cells (FIG. 4A-4B). There were small but significant differences in B-cell frequencies between recipients of ORBCEL-M and placebo at 1 and 12 months, although no clear divergence was observed in the trends over time (FIG. 4C). The proportions of dendritic cells, total monocytes, and cytotoxic NK cells were similar between the two groups throughout follow-up (FIGS. 4D-F). By contrast, the frequency of natural killer T cells, which was similar for the two groups at baseline, was significantly lower in ORBCEL-M-treated participants throughout the post infusion follow-up period (FIG. 4G).

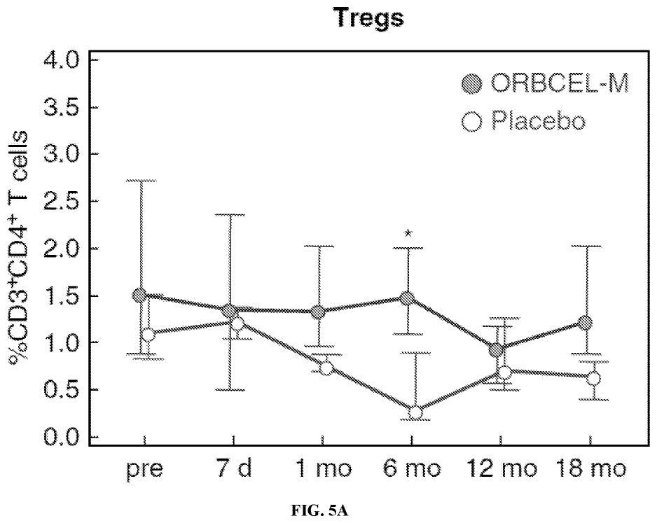

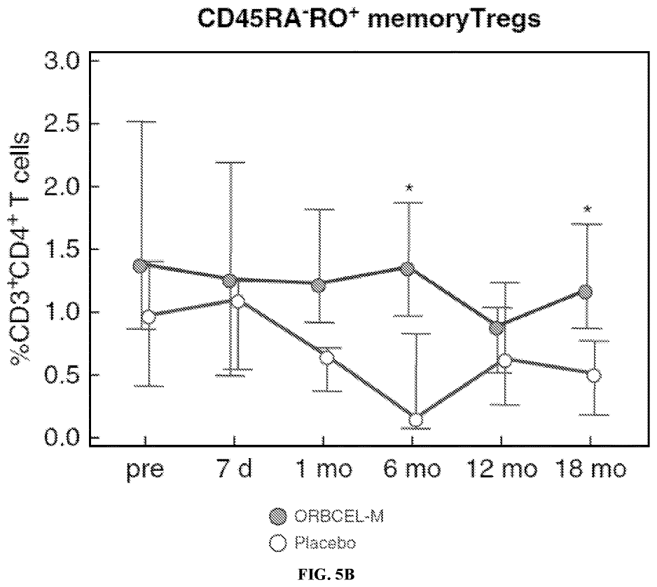

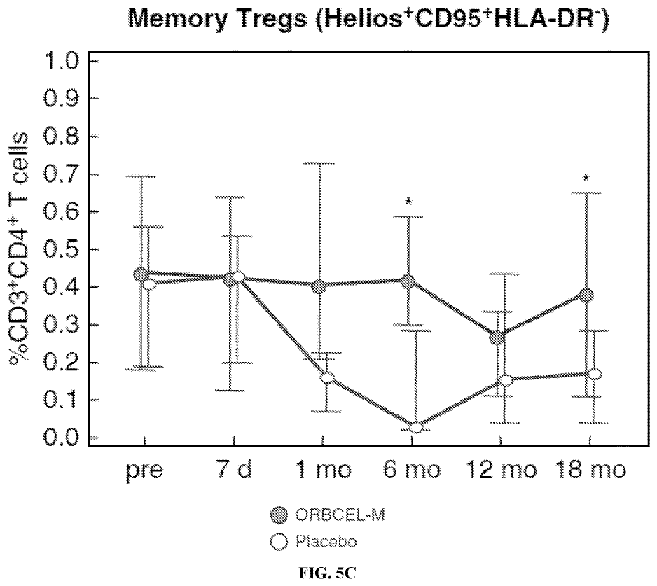

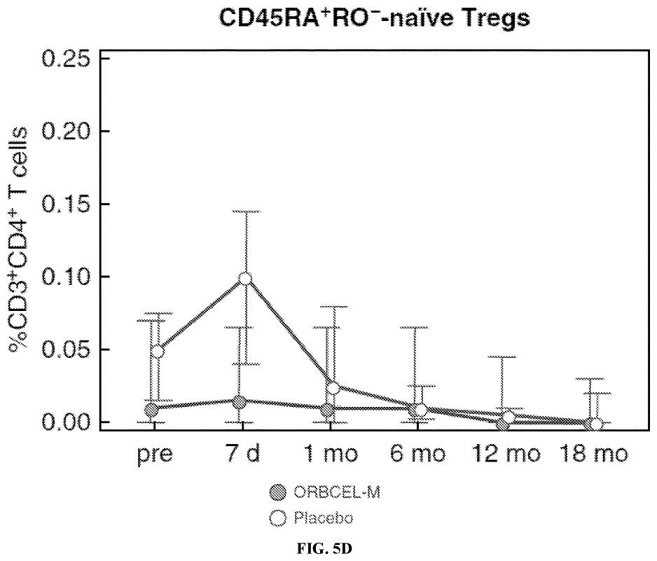

Analysis of the proportions of regulatory T cells (Tregs) among total CD3+CD4+ T cells indicated significantly higher proportions in ORBCEL-M-treated participants at 6 months (FIG. 5A). Furthermore, a subanalysis demonstrated that the proportions of memory Tregs (defined as CD45RA−RO+) declined over time in placebo-treated participants but remained stable in ORBCEL-M-treated participants—in association with significantly different proportions at 6 and 18 months (FIG. 5B). This was particularly evident for the subpopulation of memory Tregs defined as Helio S+CD95+HLA-DR−(FIG. 5C). Proportions of naïve Tregs (defined as CD45RA+RO− Tregs) remained comparably low throughout the trial in both groups (FIG. 5D).

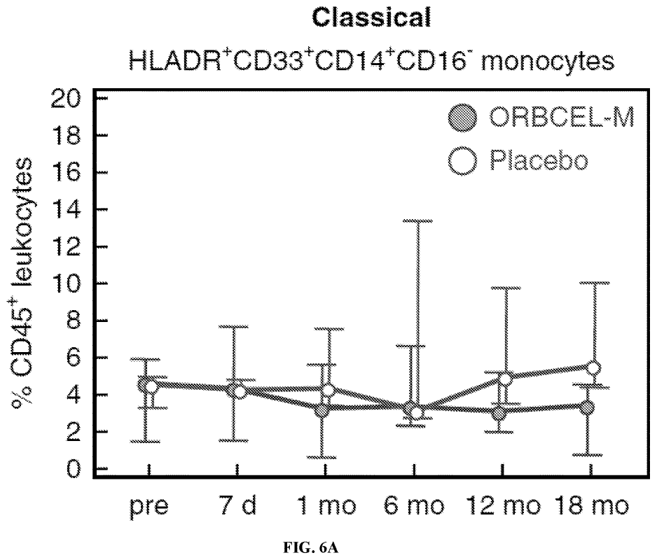

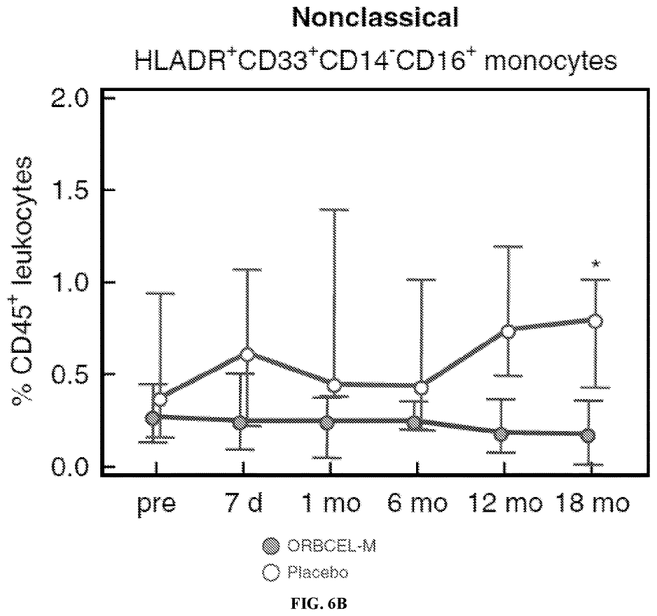

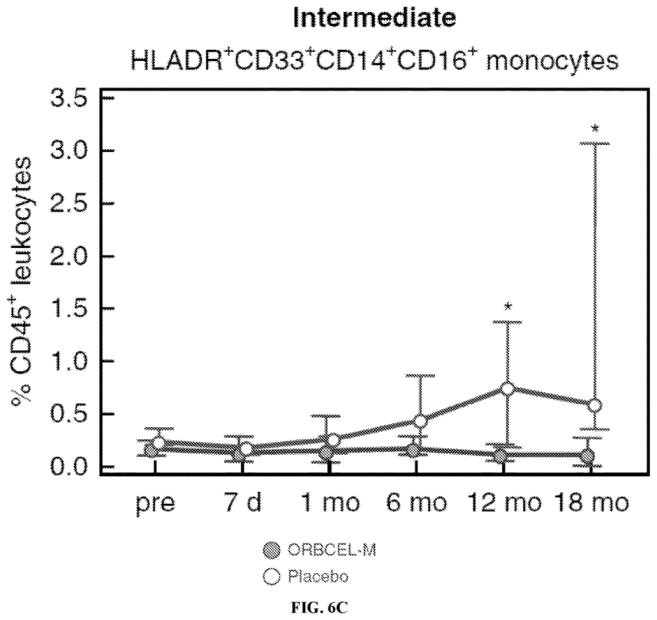

An analysis of monocyte repertoire was performed using the well-accepted 3-subset classification of classical, nonclassical, and intermediate monocytes, expressed as proportions of total CD45+ cells. As shown in FIG. 6A, the proportions of the most numerous, classical monocyte subset remained stable and very similar between the two groups through the trial follow-up period. By contrast, proportions of both the nonclassical and intermediate subsets tended to increase in the placebo-treated group between 6 and 18 months while remaining stable throughout the trial in the ORBCEL-M-treated group-being significantly lower at 18 months for nonclassical monocytes and at 12 and 18 months for intermediate monocytes (FIG. 6B, FIG. 6C).

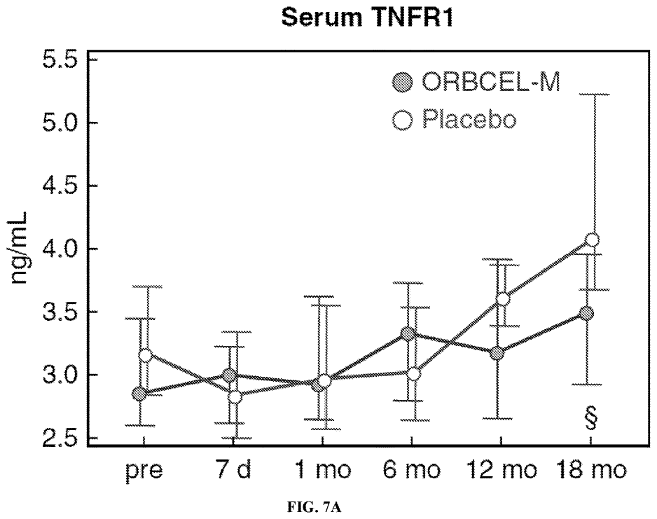

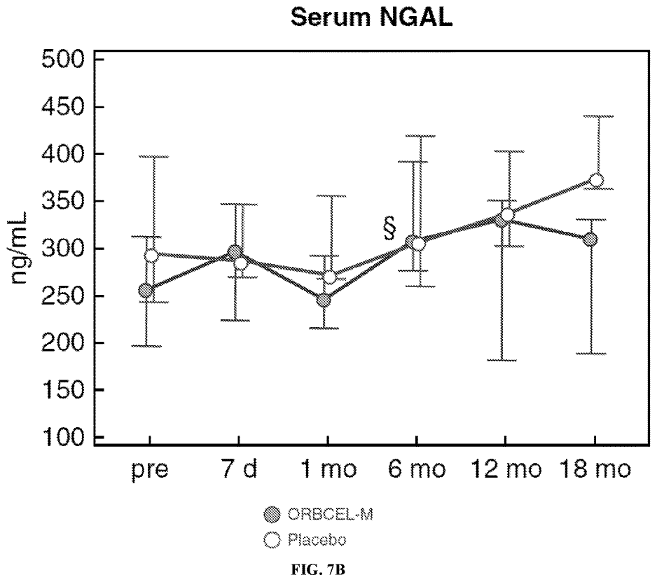

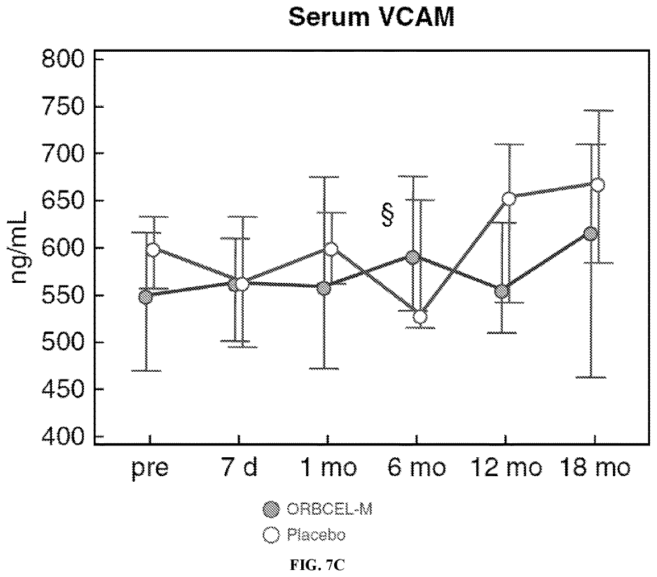

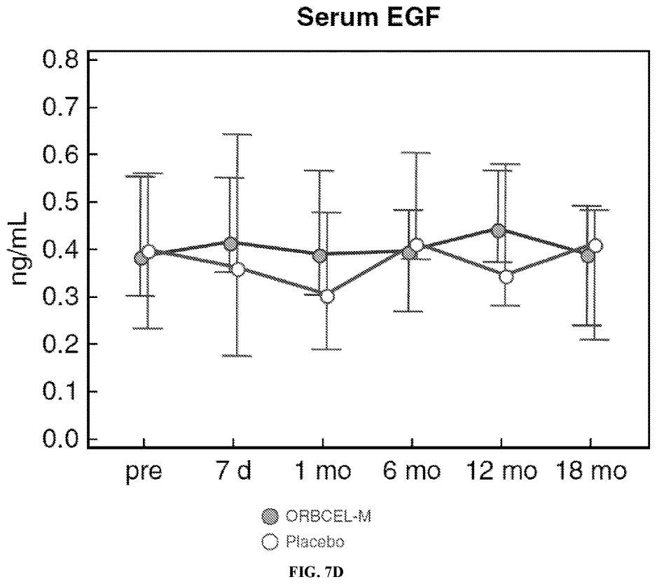

Longitudinal analyses of the serum concentrations of inflammatory biomarkers STNFR1, NGAL, and VCAM-1 indicated trends of increasing levels during the 18-month follow-up period with no between-group differences (FIGS. 7A-7C). The serum concentration of EGF remained stable in both groups (FIG. 7D).

### Other Exploratory Comparisons

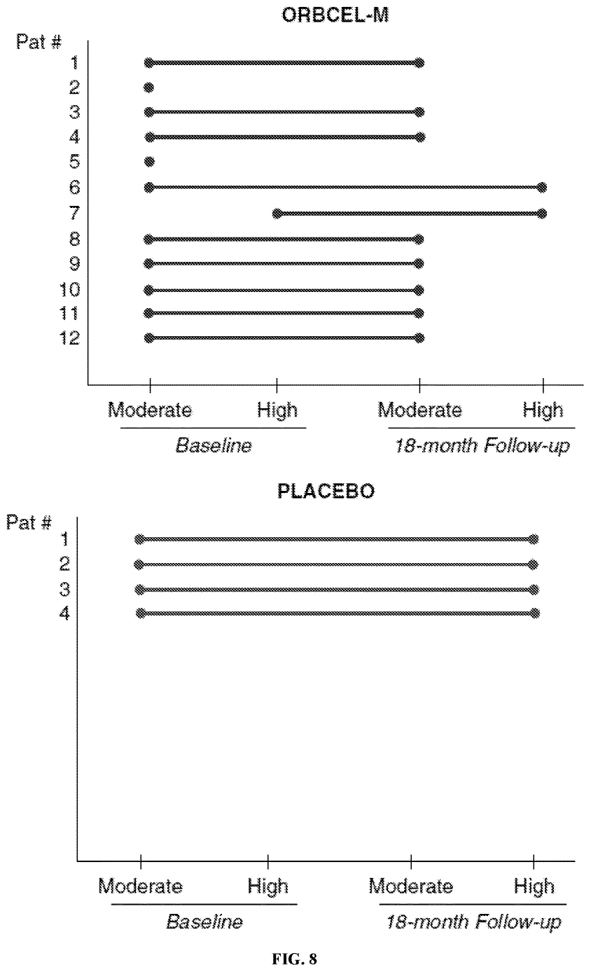

As a post hoc analysis, the progression of CKD for transitions from baseline to 18 months in the two-year risk category for reaching ESKD was compared for the two groups using the kidney failure risk equation. As shown in FIG. 8, the baseline 2-year risk category was moderate for 11 of 12 participants of the ORBCEL-M-treated group and 4 of 4 of the placebo-treated group. One participant who received ORBCEL-M was categorized as being at high risk at baseline. Of the ten participants who received cell therapy and completed 18 months of follow-up, eight remained in the moderate-risk category, one had transitioned from moderate to high risk, and one remained in the high-risk category. By contrast, all four participants who receive placebo had progressed from the moderate to high-risk category.

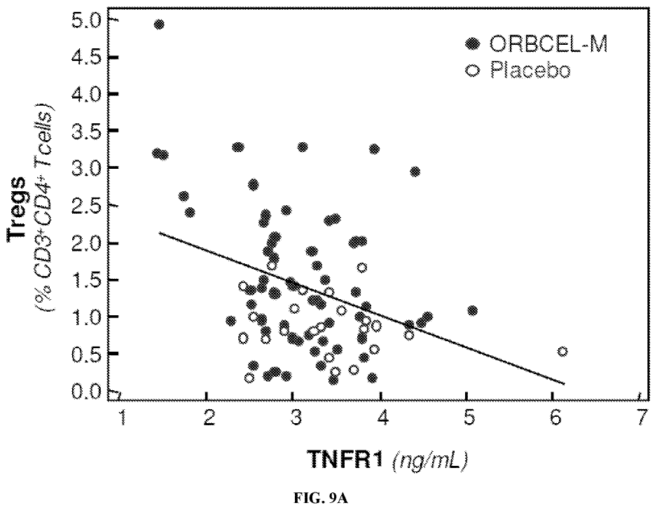

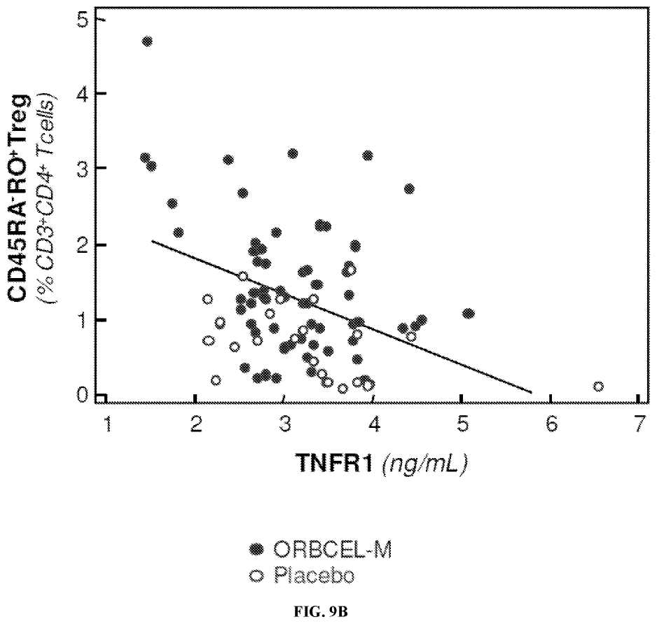

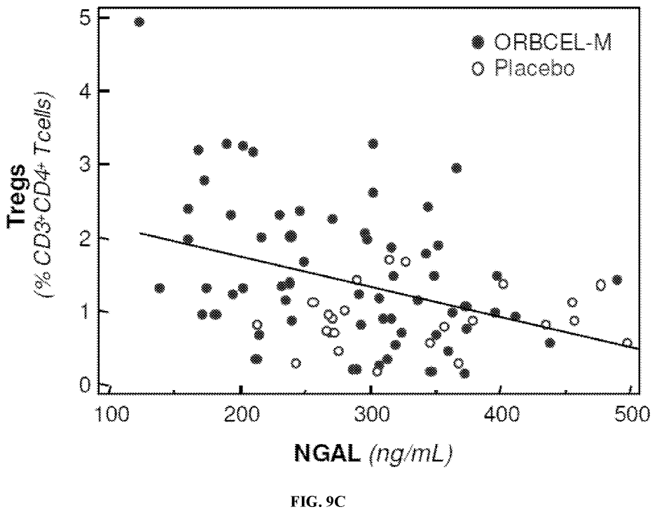

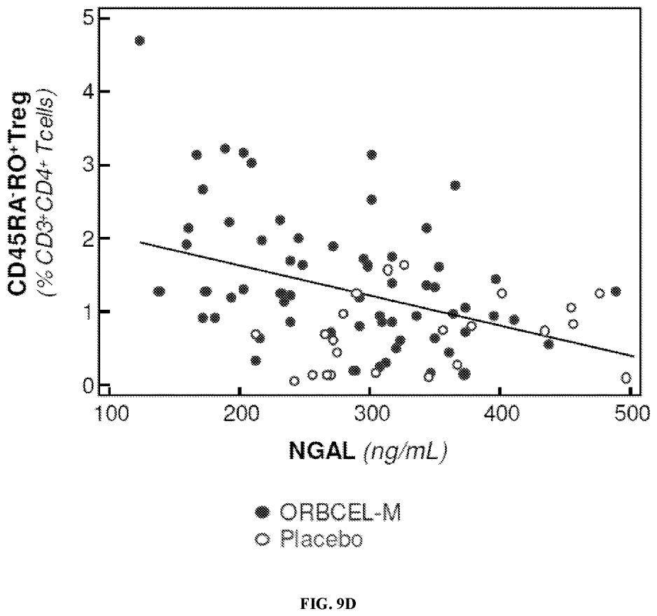

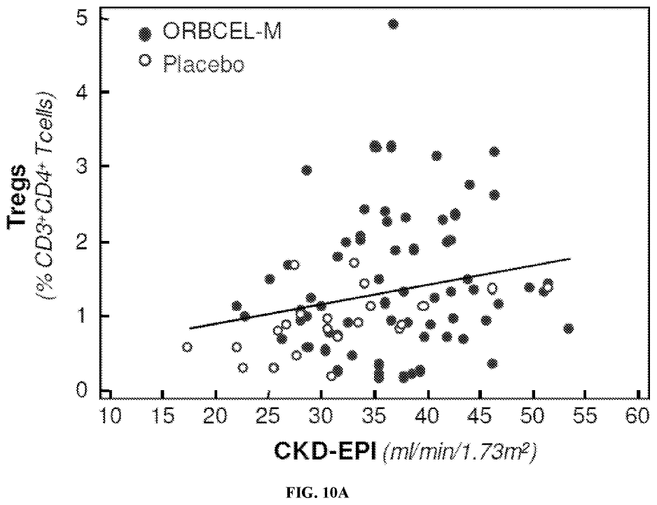

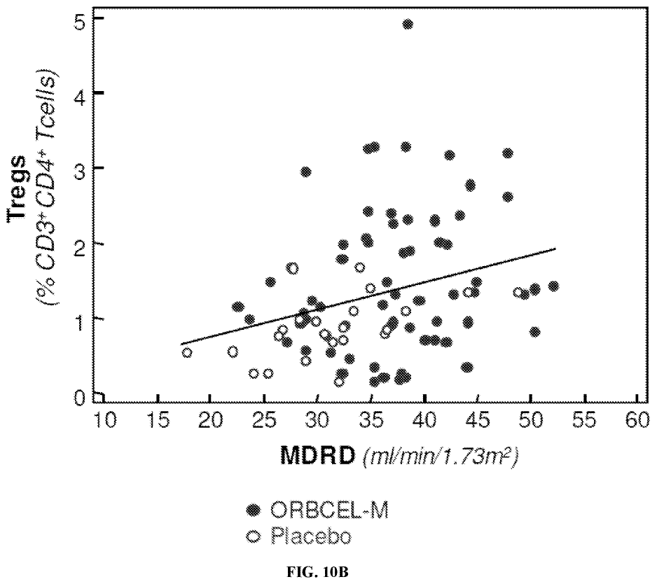

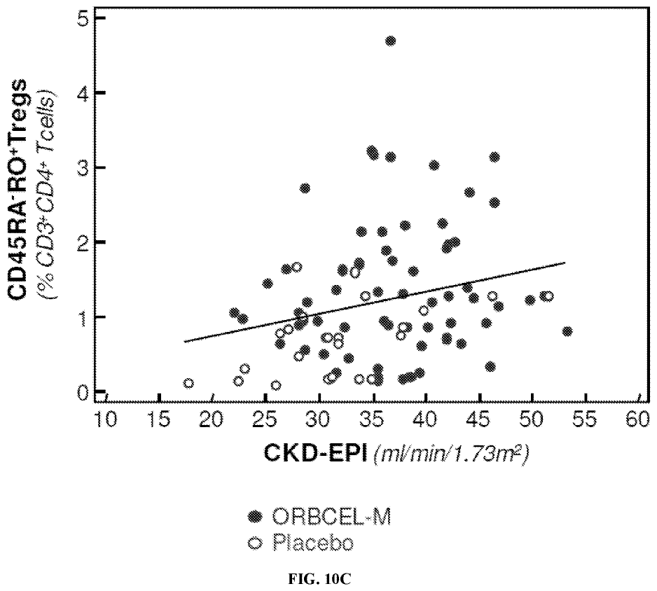

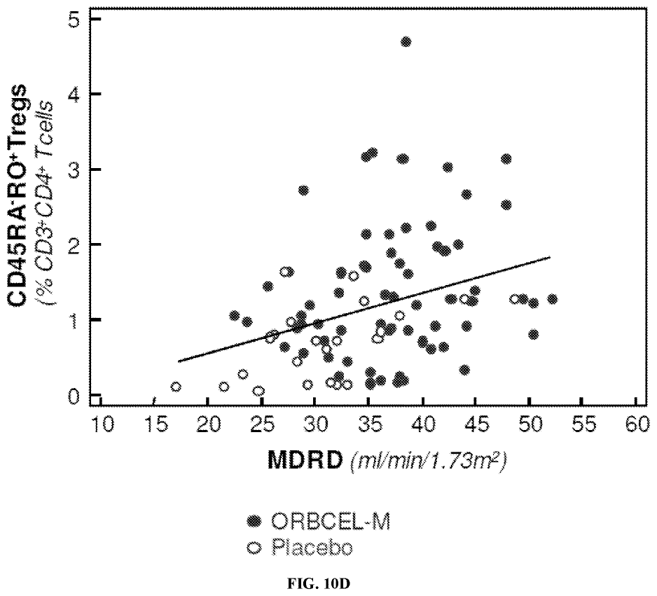

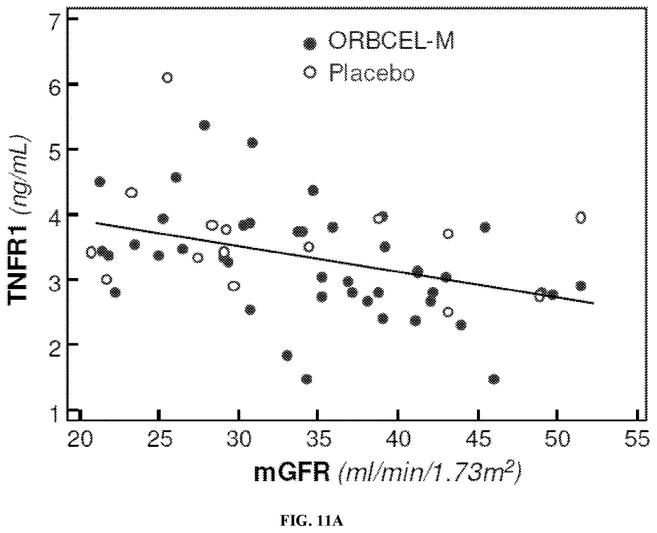

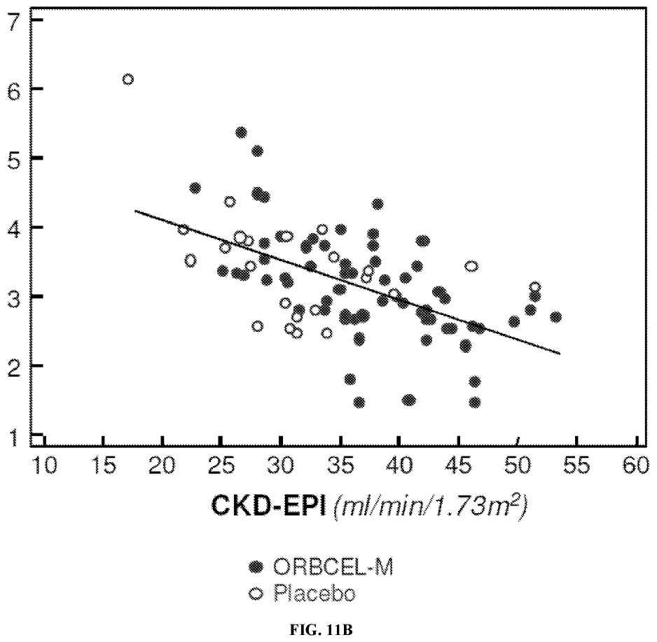

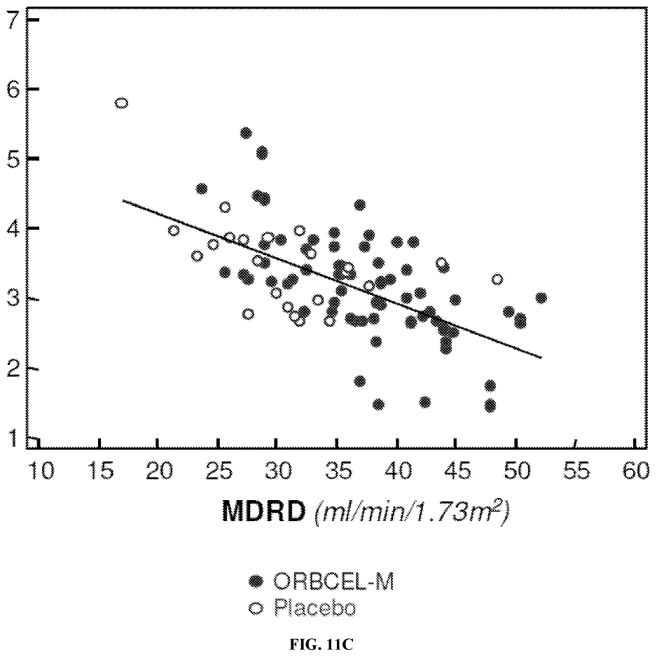

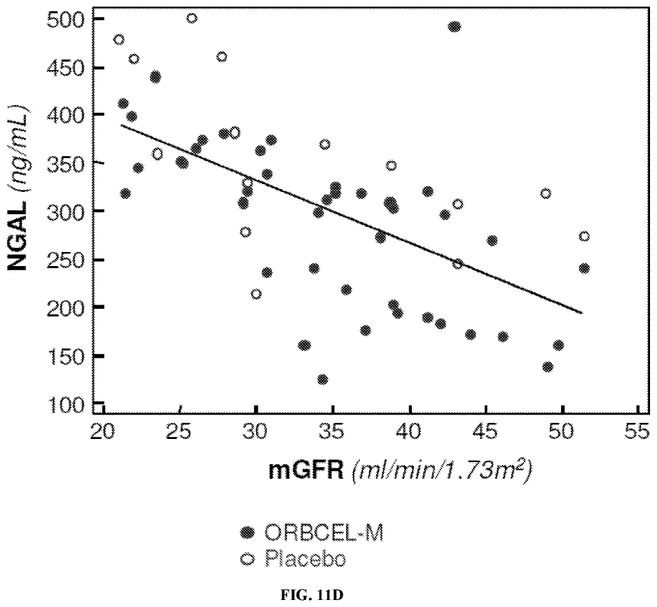

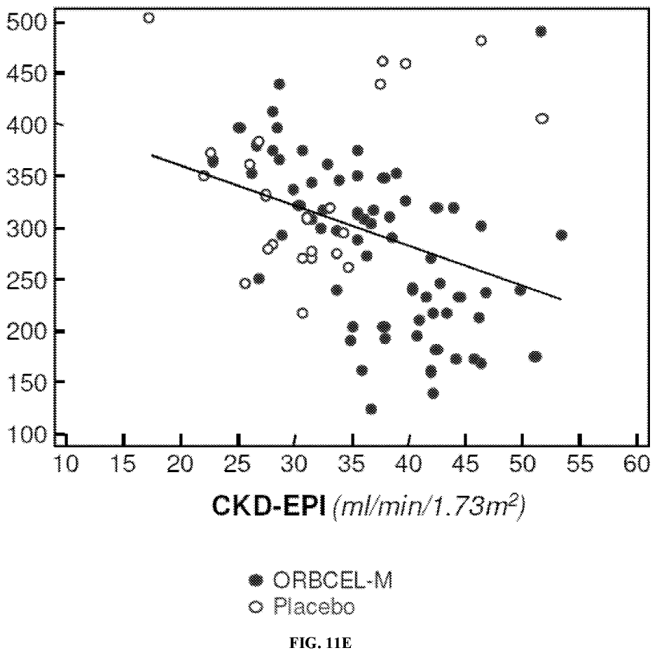

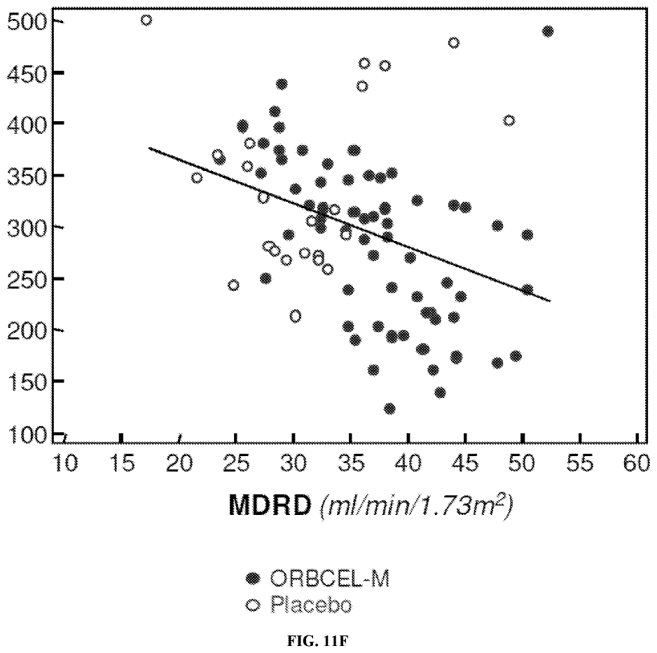

In correlative analyses of the combined immune/inflammatory profiling dataset, it was observed that peripheral blood proportions of total Tregs and CD45RA−RO+ memory Tregs for the whole cohort correlated significantly with the coincident serum concentrations of sTNFR1 (FIG. 9A, FIG. 9B). In a subgroup analysis, these correlations remained statistically significant for the group that received ORBCEL-M (P<0.002 for both cell subsets), but not for placebo-treated participants (P=0.364 and P=0.063 for Tregs and CD45RA−RO+ memory Tregs, respectively). Negative correlations were also observed between proportions of total Tregs or CD45RA−RO+ memory Tregs and coincident serum NGAL concentrations (FIG. 9C, FIG. 9D). In the cohort as a whole, there were significant positive correlations between the total Treg or CD45RA−RO+ memory Treg proportions and coincident GFR estimated by both CKD-EPI and MDRD equations (FIGS. 10A-10D). Finally, serum sTNFR1 and NGAL concentrations negatively correlated with both mGFR and eGFR (CKD-EPI and MDRD) in the overall cohort (FIGS. 11A-11F) and in the ORBCELM-treated group (sTNFR1: P<0.001 versus CKD-EPI and MDRD and P=0.003 versus mGFR; NGAL: P<0.001 versus CKD-EPI and MDRD and mGFR).

### DISCUSSION

In the first-dose cohort of this phase 1b/2a multicenter, randomized, double-blind, placebo-controlled clinical trial, it was observed that a single iv infusion of 80×10⁶ next-generation, bone marrow-derived, anti-CD362-selected, allogeneic MSCs (ORBCEL-M) was well-tolerated in participants with type 2 diabetes and progressive DKD. This acceptable safety profile was sustained in the weeks and months thereafter up to the end of the 18-month follow-up period. Importantly, SAEs, which occurred at longer time intervals after cell administration, resulted in the death of two patients. These events, however, were deemed not to be related to the trial investigational product, on the basis of the well-recognized short in vivo persistence of intravenously administered MSCs as well as the likelihood of alternative causal factors linked to medical comorbidities associated with type 2 diabetes and DKD. Indeed, the participant who died because of an acute cardiovascular event had received the cell infusion more than 14 months previously. This late time point would preclude a role for the activation of the coagulation system because rarely associated acute thromboembolic events have only been described at the end of or a few days after iv administration of MSC-based products. In this regard, a recent meta-analysis of the reported outcomes of 55 randomized clinical trials, which enrolled more than 2600 participants with a range of significant medical conditions (e.g., cardiovascular, neurological, kidney and liver diseases), showed that MSC administration was associated with an increased risk of fever, but not non-fever acute infusional toxicity, thrombotic/embolic events, malignancy, or death compared with a control group which did not receive cell therapy. None of the included clinical trials was ended prematurely because of safety concerns.

One theoretical concern with culture-expanded progenitor cell therapies, such as MSC therapy, is the possibility that such cells transform during culture and acquire the potential to give rise to tumors in the recipient after infusion. Critically, whereas malignant transformation has been demonstrated with murine MSCs, no such event has been reported for human MSC-based therapies. Furthermore, an autopsy study performed on 18 patients who had received allogeneic MSCs for hematological malignancies or solid tumors and had died between 3 and 408 days after the last MSC infusion found no ectopic tissue formation or malignant tumors of MSC origin by macroscopic or histological examination. In addition, despite their immunomodulatory properties, MSC therapies have not been associated with increased risk of developing malignancies in solid-organ transplant recipients receiving long-term immunosuppressive drugs. For the trial participant who died because of myeloma, the lack of prior reports of new or accelerated cancers among thousands of recipients of MSC therapies for diverse clinical indications (including many immunosuppressed patients with allogeneic hematopoietic stem cells transplant for bone marrow malignancies as well organ transplants and autoimmune diseases), the short duration of the culture expansion protocol for ORBCEL-M manufacture and the strict criteria for GMP release of the cell product lead us to conclude that the condition was highly unlikely to have been caused or exacerbated by the trial intervention. Nonetheless, it is possible, if not likely, that this participant had nonclinically detected plasma cell dyscrasia with circulating monoclonal protein before enrollment. This highlights the potential for non-diabetes-associated renal pathology to be present in patients participating in clinical trials for DKD (which typically relies on clinical rather than biopsy diagnosis of diabetic nephropathy) as well as the need to consider specifically screening for monoclonal gammopathy in trials of cell and other immune modulatory therapies for kidney disease.

A limited number of previously reported clinical trials have addressed the safety, tolerability, and potential benefits of MSC-based therapies in participants with type 2 DM. Such trials used autologous or allogeneic MSCs from different tissue sources; were infused into a peripheral vein or through the pancreatic artery; and, in most cases, involved cells isolated and manufactured by plastic adherence, which results in a heterogeneous/unselected stromal cell product. Extensively characterized MSC products manufactured from more selected primary tissue precursors may provide superior and more consistent therapeutic effects and may also be better suited to meet future regulatory criteria for advanced cell products. Only the early-phase trial reported by Packham et al. tested the effects of an antibody-purified allogeneic stromal cell product—specifically mesenchymal precursor cells selected for expression of the surface marker Stro3 (MPC, rexlemestrocel-L, from Mesoblast Ltd)—in participants with established DKD. The ORBCEL-M product investigated in the NEPHSTROM trial was also manufactured from primary marrow stromal cells, but was selected for expression of a different surface marker (CD362, also referred to as syndecan 2) and was culture-expanded in a hollow-fiber bioreactor such that the cells were at an early passage number when administered. The ORBCEL-M product has therapeutic benefits that are distinct from those of other stromal cell therapies tested in people with type 2 diabetes. The results reported herein indicate ORBCEL-M and rexlemestrocel-L to be equally safe and tolerable with preliminary evidence of clinically relevant efficacy. Whether ORBCEL-M may provide advantages, such as superior potency, greater efficacy, or lower cost, than other MSC-based cell therapies that have been investigated in diabetes and DKD cannot be determined until larger or comparative studies have been performed. As for the metabolic effects of ORBCEL-M, there were no notable changes in glycemic parameters (fasting plasma glucose, HbA1c) during the 18 months after cell infusion. Although preclinical and some clinical studies have suggested the potential for MSC therapy to improve glycemic control, these results are consistent with those of other early-phase trials in similar participants with type 2 DM. This may reflect, in part, the fact that the trial participants had very good glycemic control at the time of enrollment. Notably, administration of ORBCEL-M, an allogeneic cell product, proved to have minimal capacity for immunological sensitization, as evidenced by the lack of emergence of detectable anti-HLA antibodies over 18 months of follow-up in all but one participant who developed low-level anti-HLA class I antibodies from 12 months after infusion. These observations are in keeping with findings of other clinical trials which have reported that allogeneic bone marrow derived MSC products can be safely administered to humans without eliciting clinically relevant immunological reactions. Of direct relevance to the results reported herein, two reported results of clinical trials of the allogeneic mesenchymal precursor cell product, rexlemstrocel-L, in participants with type 2 DM or diabetic nephropathy, also indicated a lack of development of persistent de novo donor-specific antibodies. The observed lack of frequent or high-level sensitization against allogeneic HLA is of particular importance for the future role of ORBCEL-M, or other allogeneic MSC-derived products, in the setting of kidney disease, particularly from the perspective of the potential subsequent need for kidney transplantation. The completed 18-month follow-up of NEPHSTROM cohort 1 also showed that the rate of decline of mGFR was numerically but not significantly lower for recipients of ORBCEL-M than for recipients of placebo while similar trends for eGFR did reach statistical significance. This occurred in the setting of comparable relevant baseline characteristics and similarly acceptable metabolic and BP control between the two treatment groups. The further observation that the KRFE-based 2-year risk category for reaching ESKF worsened in all placebo-treated participants but remained stable in most of the evaluable recipients of ORBCEL-M also favors a cautious conclusion that divergent post-treatment renal functional trajectories occurred in these two groups of participants with preexisting rapidly progressive DKD. Regarding mechanism of action, MSCs are now considered to mediate their therapeutic benefits predominantly through inducible secretion of paracrine mediators and reprogramming of myeloid and lymphoid immune cells. For example, several lines of evidence have shown that MSCs promote IL-10 production by T cells through inhibition of the differentiation of Th1 and Th17 cells, thereby inducing the generation of Tregs. Furthermore, MSCs may act indirectly to promote the induction and expansion of Tregs as well as other anti-inflammatory mediators by modulating the activities of monocytes, macrophages, and dendritic cells. It is of interest, therefore, that it was observed that a divergence in the trends for circulating memory Tregs between the two trial groups—with recipients of placebo (who experienced greater rates of decline of eGFR) having diminishing proportions over time while recipients of ORBCEL-M retained more stable proportions. Although a cause-effect relationship cannot be concluded from these findings, the positive correlations observed between Tregs/memory Tregs and eGFR as well as the inverse correlations between the coincident serum concentrations of well-established DKD-associated inflammatory biomarkers sTNFR1 and NGAL and eGFR for the cohort as a whole (and in the ORBCEL-M-treated group) tend to support the hypothesis that infusion of the allogeneic MSC product is associated with a sustained immunomodulatory/anti-inflammatory effect with potential for modulating aspects of the pathophysiology of progressive DKD. Also consistent with this hypothesis is the divergence that occurred between cell therapy and placebo recipients at later time points in the trial for proportions of circulating intermediate monocytes, which have proinflammatory properties and have been linked to cardiovascular disease and rate of eGFR decline in CKD. It remains possible that clinically applicable assays of serum, plasma, and urine biomarkers linked to systemic and intrarenal inflammation or fibrosis will provide value as indicators of response to MSC therapy. However, the interindividual variability that was observed for selected serum biomarkers in this cohort suggests that larger participant numbers will be required to meaningfully investigate this question. Finally, it should be acknowledged that the potential anti-inflammatory/immune-modulating effects observed in recipients of ORBCEL-M and documented in many other preclinical and clinical studies bring at least a theoretical risk of promoting or worsening infection and cancer and that this potential must continue to be investigated in a careful and unbiased manner in trials such as ours.

In conclusion, the results reported here for a completed cohort of the multisite, randomized, double-blind, placebo-controlled NEPHSTROM trial document the safety and tolerability of a single infusion of 80×10⁶ ORBCEL-M. In addition, these findings confirm low potential for this MSC-based cell therapy product to sensitize recipients against allogeneic HLA and reveal preliminary evidence for potential reno-protective and immune modulatory effects over an 18-month post-infusion observation period. These results, as well as the continued need for new, disease-modulating therapies to preserve renal function in people with progressive DKD support further investigation of ORBCEL-M in an appropriately sized and powered phase 2b study.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of treating a diabetic kidney disease in an individual, the method comprising administering a single dose of about 80×10⁶ SDC2+ stromal cells to the individual, thereby treating the diabetic kidney disease, wherein the single dose is sufficient to improve kidney function compared to an untreated individual.

2. The method of claim 1, wherein the kidney function improvement is evaluated by measuring glomerular filtration rate (mGFR) or estimating glomerular filtration rate (eGFR).

3. The method of claim 2, wherein measured glomerular filtration rate (mGFR) or estimated glomerular filtration rate (eGFR) is improved compared to a baseline measurement.

4. The method of claim 2, wherein measured glomerular filtration rate (mGFR) or estimated glomerular filtration rate (eGFR) does not decline compared to a baseline measurement.

5. The method of any one of claims 1 to 4, wherein the single dose treatment is effective to improve the kidney function for at least 18 months.

6. The method of any one of claims 1 to 5, wherein the single dose treatment prevents kidney failure for at least 18 months.

7. The method of any one of claims 1 to 6, wherein frequency of regulatory T cells (CD4⁺CD25^{high}FoxP3⁺CD127⁻) is increased after the single dose treatment in the individual compared to an untreated individual.

8. The method of any one of claims 1 to 7, wherein frequency of memory regulatory T cells (measured by CD4⁺CD25^{high}FoxP3⁺CD127⁻CD45RA⁻CD45RO⁺ or CD4+Helios⁺CD95⁺HLA-DR⁻) is increased after the single dose treatment in the individual compared to an untreated individual.

9. The method of any one of claims 1 to 8, wherein the frequency of natural killer T (NKT) cells (CD3+CD56+) does not increase compared to an untreated individual.

10. The method of any one of claims 1 to 9, wherein the frequency of intermediate activated monocytes (HLADR⁺CD33⁺CD14⁺CD16⁺) and/or non-classical patrolling monocytes (HLADR+CD33+CD14−CD16+) is suppressed or decreased after the single dose treatment in the individual compared to an untreated individual.

11. The method of any one of claims 1 to 10, wherein serum cytokines NGAL and sTNFR1 are decreased after the single dose treatment in the individual compared to an untreated individual.

12. The method of any one of claims 7 to 11, wherein the frequency of regulatory T cells, memory regulatory T cells, NKT cells, intermediate activated monocytes and/or non-classical patrolling monocytes is observed for about 12 months to about 18 months after treatment.

13. The method of any one of claims 1 to 12, wherein the individual has type 2 diabetes or suffering from a symptom of type 2 diabetes.

14. The method of any one of claims 1 to 13, wherein the individual has urinary albumin excretion (UAE) of at least 60 μg/min prior to the treatment.

15. The method of any one of claims 1 to 14, wherein the individual has urine albumin-to-creatinine ratio (UACR) of at least 88 mg/g or 10 mg/mmol prior to the treatment.

16. The method of any one of claims 1 to 15, wherein the individual has an eGFR of at least 30 to 50 ml/min/1.73 m² prior to the treatment.

17. The method of any one of claims 1 to 16, wherein the individual has experienced a decline in eGFR of at least 10 ml/min/1.73 m² over three years prior to the treatment.

18. The method of any one of claims 1 to 17, wherein the individual has experienced a decline in eGFR of at least 5 ml/min/1.73 m² over 18 months prior to the treatment.
